(19) United States
(12) Patent Application Publication
(10) Pub. No.: US 2010/0009858 A1
(43) Pub. Date: Jan. 14, 2010

(54) EMBRYONIC STEM CELL MARKERS FOR CANCER DIAGNOSIS AND PROGNOSIS

(75) Inventor: Chunde Li, Sodertalje (SE)

Correspondence Address: DICKSTEIN SHAPIRO LLP, 1633 Broadway, NEW YORK, NY, 10019, US

(73) Assignee: CHUNDSELL MEDICALS AB, Stockholm (SE)

(21) Appl. No.: 12/375,177

(22) PCT Filed: Jul. 16, 2007
(86) PCT No.: PCT/SE07/00689

§ 371 Date: Jan. 26, 2009

(30) Foreign Application Priority Data
Jul. 28, 2006 (SE) 0601615-8

(51) Int. Cl. C40B 30/00; C40B 40/06; C07H 21/02; C07H 21/04

(52) U.S. Cl. 506/7; 506/16; 536/24.31

(57) ABSTRACT

A method of predicting the development of a cancer in a patient, comprises procuring a sample of tumour tissue from the patient, determining the expression pattern of embryonic stem cell genes in the tissue, comparing the expression pattern with the corresponding expression pattern of embryonic stem cell genes in tumour tissue of reference patients with known disease histories. Also disclosed are microarrays and DNA/RNA probes for use in the method.

### FIELD OF THE INVENTION

The present invention relates to embryonic stem cell (ES) gene markers for use in diagnosis and prognosis of cancer, in particular prostate cancer.

### BACKGROUND OF THE INVENTION

Gene expression profiling in cancer cells of various kind as well as in embryonic stem (ES) cells using high throughput DNA microarrays is known in the art. A direct link between tumor and ES cell expression signatures has however not been established.

Bioinformatic analyses based on published or unpublished high throughput proteomic data have not yet reached robust and high resolution as compared with high throughput DNA and RNA analyses. Bioinformatic analyses based on published and unpublished high throughput genome-scale DNA analyses provide a list of DNA markers in the form gene copy number changes (deletions, gains and amplifications), mutations and polymorphisms, and methylations. DNA is comparatively stable and easy to be handled in analytical process. However, these DNA changes have to be detected by different methods.

It is still an open question why cancer originating from the same kind of tissue progresses slowly in one person and rapidly in another. Recent expression profiling analyses have provided quite complete and specific molecular portraits of many cancers, especially of subtypes of a particular cancer differing in clinical outcome (1-4). Some studies even provided short lists of genes, the expression of which is predictive of the outcome of the respective cancer (5-6). These expression profiling results have led to further functional studies of selected markers or genes (7). However, in general, the selection of “important” genes is based on a pure statistical approach (8-9). Despite many new theories and methods trying to coup with the challenge of huge amounts of data-provided by high throughput experiments, the statistics in this field is still very much under development. Most studies therefore turn into a lottery from a list of “markers”, and their result is largely confined to a molecular phenotypic level (10).

Prostate cancer is a major cause of death worldwide in male adults. Accurately predicting the outcome of prostate cancer at an early stage of tumor development is crucial for providing the proper kind of treatment, and is still an unresolved question. The correct choice of treatment is most important in younger patients (11). It is estimated that of 232,090 American men with newly diagnosed prostate cancer in 2005, roughly 210,000 or approximately 90% will be diagnosed at an early stage with 100% survival for 5 years. In contrast, the estimated deaths from prostate cancer are much less, about 30,350 (12). Online data from the Swedish National Board of Health and Welfare have shown that 7,702 out of 4,427,107 Swedish men in 2001 had newly diagnosed prostate cancer. In a randomized clinical observation of 348 patients with early stage and well to moderately-well differentiated prostate cancer, 108 (31%) showed local progression, 54 (15.5%) had distant metastases and only 31 (8.9%) had deceased from prostate cancer after 8 years follow-up (13). Some early stage prostate cancers can be indolent during 8 years of follow-up and display accelerated progression later after a follow-up of more than 15 years. However, these late-progressive tumors only constitute up to 17% of all early stage cases (14). Current clinical diagnostic and prognostic methods can not accurately distinguish this small group of early stage cancer with aggressive potential from the more common less-aggressive early stage tumors (15).

Humphrey P A has given a comprehensive review of Gleason grading and current status of clinical methods in diagnosis and prognosis of prostate cancer (15-16). Today, the Partin Table is the most widely used method for choosing proper treatment (17-18) integrating important clinical parameters to predict the pathological stage. Important parameters are Gleason score of needle core biopsy, serum PSA level and clinical stage. Of all parameters, cytological grade or Gleason grading of biopsy samples is currently the key method for confirming the diagnosis of prostate cancer, and has demonstrated strong association with cancer specific survival. However, Gleason grading is not satisfactory for predicting cancer outcome when tumors are small, in particular when tumors are moderately differentiated with a biopsy Gleason score 6, the most common Gleason sum in clinical biopsy cases (15). Quite often, a diagnosis of prostate cancer is uncertain due to insufficient, or lack of, malignant structures, rendering further prediction of cancer outcome impossible (15). Waiting time for capturing confirmative malignant structure by repeated biopsy procedures may miss the right time window to cure patients with life-threatening cancer at very early stage. On the other hand, uncertain outcome prediction causes reduction of life quality in patients with virtually harmless cancer when they are treated with radical surgery. There is currently a strong need for a new diagnostic and prognostic method that can complement and improve Gleason grading system in three aspects (19): firstly, it should directly reflect biological aggressiveness, i.e. be able to predict different outcome of tumors with the same Gleason grade, in particular tumors with Gleason score 6; secondly, it should apply to small biopsy samples; thirdly, it should be able to predict tumor aggressiveness using biopsy samples from cancerous prostate with insufficient malignant structure, overcoming problems with small tumors and heterogeneous tumors that limit the accuracy of histopathological evaluation of biopsy samples.

An abundance of experimental data shows that cancer is caused by genomic alterations. Weinberg R A and associates as well as Vogelstein S and associates reviewed these data and developed them into generally accepted theories of the molecular genetics and biology of cancer (20-26). Briefly, the genomic changes involved include DNA sequence changes, such as base change, deletion, copy number gain, amplification and translocation, as well as DNA modification such as promoter methylation. These genomic changes cause gene expression alterations that further cause biological alterations in the cell, such as accelerated cell cycle, alteration of cell-cell contact and signaling, increase of genomic instability, escape from apoptosis, increase of cell mobility, activation of angiogenesis and escape from immune surveillance. It has been shown that five to six genomic alterations are needed to establish a malignant phenotype of invasion and metastasis, meaning that multiple biological functional alterations are required. Different initial and subsequent key genomic events may determine different potential of invasion and metastasis, a basis for using molecular genetic markers to predict clinical outcome of cancer (20-26). So far, only a few genetic or epigenetic alterations have been identified in prostate cancer at individual gene level, such as germline mutations of RNASEL (HPC1) and ELAC2 (HPC2) in patients with hereditary prostate cancer, somatic mutations of PTEN, EPHB2 and AR in sporadic prostate cancer, and promoter methylation of GSTP1 in prostate cancer tissues (27-34). Nelson W G, De Mazo A and Isaacs W B have concisely reviewed the current status of prostate cancer molecular genetic and biological studies (11; 35-36). Tricoli J V and associates have summarized all putative diagnostic and prognostic markers of prostate cancer (19). An important question remains: no single molecular biomarker has turned out to be superior to the Gleason grading system. This is due to the fact that Gleason grading is a morphological profiling indirectly reflecting most important biological alterations, whereas a single biomarker may merely reflect alterations of one or two biological pathways in cancer cells. The broad spectrum of tumor genotype alterations and phenotype variations has hindered successful translation of findings from most single marker analysis into useful clinical markers for predicting disease outcome.

In contrast, high throughput methods such as DNA arrays allow profiling of molecular signatures indicating alterations of multiple cellular processes (37). There is an increasing body of studies of using gene expression profiling to extract specific expression patterns or signatures attributed to different biological forms of cancer, and further using these gene expression features to predict clinical outcome of early stage cancer, e.g. breast cancer (5; 6). There are also several publications on gene expression profiling of human prostate cancer (1; 7; 38-54). Their quality differs by array complexity, number of cases and tissue samples studied, but they share two limitations: (i) they used a small number of cases selected by surgery with short time follow-up; (ii) antibody availability limited the use of immunohistochemistry to verify clinical importance of most new genes in a large series of tissue arrays. Proteins as markers do not always reflect RNA alterations.

Despite these disadvantages, previous studies have identified several new markers that are potentially useful in clinics, such as AMACR in distinguishing cancer from non-cancer lesions, HPN, PIM1 and EZH2 in prognosis, as well as AZGP1 and MUC1 in distinguishing different forms of primary tumors. However, none of these markers is superior to Gleason grading.

In earlier co-operative work with Stanford University the present inventor carried out gene expression profiling in a large set of normal prostate tissues, prostate tumors and lymph node metastases. Using various statistical approaches, a few hundreds genes were identified, the expression of which allows to distinguish low grade from high grade tumors, and even to predict the risk of short-term recurrence after radical surgery. High throughput tissue microarray analysis with a series of selected markers has found that MUC1 showed significant increased expression in tumors with poor prognosis and AZGP1 showed increased expression in tumors with good prognosis. However, even the two markers in combination do not have the same predictive power as histopathological evaluation using the Gleason grading system. This indicates the limitation of this marker lottery approach (1).

Thus, with the advancement of biological and genetic research, knowledge about initiation and progression of cancer has greatly increased in recent time. Successful use of such knowledge in clinical diagnosis, prognosis and treatment for cancer patients, however, has been limited so far.

A highly relevant problem is how to predict the outcome of a tumor in a patient. Predictive methods available today are based on the concept that all tumor cells in a specific tumor are of the same functional importance. New data has shown that the total tumor cell population can be divided into two populations, i.e., a small tumor stem cell population and a large partially differentiated tumor cell population. Tumor stem cells are malignant cells that can proliferate, invade and metastasize, whereas differentiated tumor cells do not possess these properties.

Most conventional methods in this field rely on one or a few tumor markers only for diagnosis and prognosis. Tumor initiation and progression is however a complex biological process involving multiple genetic and functional changes in the tumor stem cells, which can not be simply reflected by one or a few tumor markers. Therefore using one or a few tumor markers to predict tumor outcome cannot reach a level of accuracy required by clinicians and patients for proper choice of treatment alternatives. On the other hand, the indiscriminate use of all tumor markers available in a prediction method results in high experimental and methodical complexity, and thus is time consuming and costly. It is this deficiency that the present invention seeks to remedy.

### OBJECTS OF THE INVENTION

It is an object of the invention to provide a method for predicting the development of cancer at an early stage of tumor development.

It is another object of the invention to provide a method for identifying, in a group of persons diagnosed to have a cancer, a sub-group of persons in which the cancer should be treated.

It is a further object of the invention to provide a method for assigning a suitable treatment to a person pertaining to a group of persons in which the cancer should be treated.

Still further objects of the invention will become evident from the study of the following description of the invention and a number of preferred embodiments thereof, and of the appended claims.

### SUMMARY OF THE INVENTION

The present invention is based on the concept that a method for predicting the development of cancer should be based on the genetic profile of tumor stem cells, notwithstanding that they do comprise only a small portion of the total tumor cell population.

Embryonic stem cell (ES) gene markers of the invention are herein referred to as ES tumor predictor genes (ESTP genes). The gene symbols for the ESTP genes of the invention are given according to their standard symbols in the National Center for Biotechnology Information's gene database (http://www.ncbi.nlm.nih.gov/entrez/query.fcgi?db=gene&cmd=search&term). For expressed sequence tag (EST) without gene symbol, the IMAGE clone ID or the UniGene cluster ID is given.

The present invention is further based on the concept that embryonic stem cells are the origin of all tissue cells including so called progenitor cells of various specific cell lineages or cell types. Tumor cells may be derived from a few tissue stem cells whose regulatory system to guide time- and space-specific differentiation is disabled due to incorrectly repaired DNA damage. Despite impaired differentiation, other stem cell functional properties are more or less maintained or even enhanced, such as proliferation and metastasis. Thus, the more stem cell properties are conserved in the tumor cells, the more aggressive they will be biologically and clinically.

Based on this hypothesis a series of published original datasets in the Stanford Microarray Database (SMD) was analyzed according to the present invention. The datasets are derived from gene expression profiling studies in embryonic cell lines and cancers of the prostate, breast, lung, brain, stomach, kidney, ovary and blood. The expression profile of ESTP genes, that is, genes strongly regulated in ES tumor cells, allows to predict histological as well as biological subtypes with different clinical outcomes. In this application, “strongly regulated” applies to ESTP genes with a specific high expression level but also to ESTP genes with a specific low expression level.

Thus the present invention is additionally based on the hypothesis that strongly regulated ESTP genes in ES tumor cells, play a crucial role in tumor development and that, more specifically, different patterns of expression alterations of these ESTP genes determine tumor aggressiveness. According to the present invention this hypothesis is validated by using a large series of published datasets of genome-wide gene expression profiling in ES cells and in normal and tumor tissues for identifying ES genes of high prognostic power, that is, ESTP genes:

By a simple one class ranking test method, a list of 641 genes was identified, of which 328 display with highest level of expression and 313 with lowest level of expression in ES tumor cells (p≦0.05). The gene expression data of these ESTP genes were derived from a variety of normal and tumor tissue samples, in total about 1000 tissue samples (arrays). They can be used to predict pathological and clinical characteristics of a tumor in a patient by applying a simple hierarchical cluster method to a corresponding dataset obtained for the respective tumor. By this method high prognostic accuracy was obtained for all tumor types investigated, in particular prostate cancer but also gastric cancer, lung cancer, and leukemia. Moreover, prognostic accuracy was also obtained for breast cancer, ovary cancer, brain tumor, soft tissue tumor, and kidney cander.

Most important, according to the present invention, prognostic analysis is based on the genes with highest and lowest level of expression, that is, genes within ranges of expression which are near or comprise the level of maximal expression and of minimal expression.

Identification of pathological and clinical tumor characteristics by the ES gene expression profile of a tumor according to the present invention is competitive with and may be even superior to that obtained by complex statistical methods known in the art using the original expression datasets in a complete genome-wide scale analysis comprising over 20,000 genes. The present invention provides a prognostic method of predicting tumor pathological and clinical characteristics in a patient based on a restricted number of ES genes, such as less than 2,500 ES genes, more preferred less than 1,000, even more preferred from 500 to 750 ES genes, in particular from 600 to 680 ES genes, most preferred about 641 ES genes. The relatively small number of ES genes used for prediction, such as about 641 ES genes, and their specific functionality in stem cell biology allows errors due to biological and methodological background noise to be reduced or even eliminated. Virtual experimental methods based on such a restricted number of ES genes can be used for the diagnosis and prognosis of a broad spectrum of tumors. In contrast methods known in the art usually rely on few markers restricted to different tumor types. Based on the ESTP genes of the invention, a variety of robust analytical methods can be designed and applied in tumor diagnosis and prognosis using trace amounts of RNA derived from small tumor samples. For most tumors, such as prostate cancer, there is no method known in the art capable of predicting with good accuracy clinical outcome at an early stage of tumor development. It is in particular here that the prognostic method of the invention solves an important clinical problem.

In the following are disclosed preferred aspects of limiting the number of ESTP genes on which the method of the invention is based.
(I) A first preferred aspect comprises selecting ES genes of predictive significance, that is, ESTP genes that constitute a minor proportion of all ES genes, in a cancer;
(II) According to a second preferred other statistical methods can be applied to derive substantially similar ES genes for the prediction of tumor pathological and clinical characteristics as described above;
(III) According to a third preferred aspect of the invention genes with weak prediction power are eliminated from the list of ES genes identified by the method of the invention and thus from consideration, thereby reducing the number of ESTP genes and improving prediction accuracy;
(IV) According to a fourth preferred aspect of the invention a number of ESTP genes with high specificity are selected from the ES gene list obtained by the method of the invention for application to a specific type of tumor, such as prostate cancer or breast cancer;
(V) According to a fifth preferred aspect of the invention methods known in the art used in diagnosis and prognosis of tumors are based on one or several ESTP genes identified by the method of the invention, such as multiplex or high throughput RT-PCR (reverse transcriptase polymerase chain reaction) using small amounts of tumor samples, a specific DNA microarray platform, and other low or high throughput RNA analytical methods.

FNA (Fine Needle Aspiration) biopsy for clinical diagnosis and prognosis allows sampling multiple areas to cover a large volume of a tumor due to its minimal morbidity, thus being superior in overcoming tumor heterogeneity. Once the needle is inserted into a tumor lesion, it allows to obtain very pure cytological aspirates from the tumor with minimal stromal or normal epithelial cell contamination. FNA biopsy is a preferred method for obtaining pure tumor samples for molecular diagnosis and prognosis from small tumors, in particular from early stage prostate tumors. Conventional cDNA array experiments require approximately 40 μg total RNA. FNA biopsy yields 100-2,000 ng total RNA (57-59). This small amount of RNA is sufficient for analyses by using a small array platform as well as by multiplex or other high throughput RT-PCR methods.

Thus, according to the present invention is disclosed a method of predicting the development of a cancer in a patient, comprising:
(i) procuring a sample of tumour tissue from the patient;
(ii) determining the expression pattern of embryonic stem cell genes in the tissue;
(iii) comparing said expression pattern with the corresponding expression pattern of embryonic stem cell genes in tumour tissue of reference patients with known disease histories.

According to the present invention is disclosed, in particular, a method of predicting the development of a cancer in a patient, comprising:
(a) procuring a tumour tissue from the patient;
(b) determining an expression pattern of embryonic stem cell genes listed in Table 1;
(c) comparing said expression pattern with a corresponding expression pattern of embryonic stem cell genes in tumour tissue of reference patients with known disease histories;
(d) identifying the patient or patients with known disease histories whose expression pattern optimally matches the patient's expression pattern;
(e) assigning, in a prospective manner, the disease history of said patient(s) to the patient in which the development of cancer shall be predicted.

It is preferred for the determination of the expression pattern of said embryonic stem cell genes to comprise that of a first group genes with high level of expression and that of a group of genes with a low level of expression, said first and second group of genes not comprising by a third group of genes with intermediate levels of expression.

It is particularly preferred for the genes in the first group and/or the second group to be consecutive, that is, ranked consecutively, in respect of their expression levels.

According to a preferred aspect of the invention it is preferred for the total number of genes in the first and second groups to be substantially smaller than the number of the genes in the third group, in particular less than a fifth of the number of the genes in the third group. The total number of genes in the first and second groups is preferably from 500 to 750, more preferred from 600 to 680, most preferred about 641.

The genes pertaining to the first and second groups are preferably identified by employing a q value of from 0.01 to 0.1, more preferred of from 0.025 to 0.075, most preferred of about 0.05, in a one class significant analysis of microarrays (SAM) on a centered embryonic stem cell gene dataset by which all genes are ranked according to their expression levels

The method of the invention is applicable to cancer of any kind, in particular to prostate cancer, gastric cancer, lung cancer, and leukemia.

According to a second preferred aspect of the invention is disclosed the use of an embryonic stem cell gene DNA or RNA microarray for predicting the development of a cancer tumor in a patient. Preferably the microarray comprises DNA or RNA of a first group of embryonic stem cell genes with high level of expression in the tumor and of a second group of embryonic stem cell genes with a low level of expression in the tumor but not comprising DNA or RNA, respectively, of embryonic stem cell genes with an intermediate level of expression in the tumor. It is also preferred for the genes in the first and second groups to be those ranked according to their expression levels, in particular in a consecutive manner. A preferred method of ranking is a one class significant analysis of microarrays (SAM) on a centered embryonic tumor stem cell gene dataset by employing a q value of from 0.01 to 0.1, more preferred of from 0.025 to 0.075, most preferred of about 0.05. The embryonic stem cell gene DNA or RNA microarray can be used for the predictions of the development of any cancer, in particular of prostate cancer, gastric cancer, lung cancer, and leukemia and, furthermore, of breast cancer, ovary cancer, brain tumor, soft tissue tumor, and kidney tumour.

According to a third preferred aspect of the invention is disclosed a microarray comprising a fragment of embryonic stem cell gene DNA or RNA derived from a first group of embryonic stem cell genes with high level of expression in a cancer tumor and from a second group of embryonic stem cell genes with a low level of expression in said cancer tumor but not comprising a fragment of embryonic stem cell gene DNA/RNA with an intermediate level of expression in the tumor. It is particularly preferred for the genes in the first group and/or the second group to be ranked consecutively in respect of their expression levels. It is preferred for the genes in the first and second groups to be those ranked according to their expression levels by a one class significant analysis of microarrays (SAM) on a centered embryonic tumor stem cell gene dataset by employing a q value of from 0.01 to 0.1, more preferred of from 0.025 to 0.075, most preferred of about 0.05. The cancer can be any cancer, in particular prostate cancer, gastric cancer, lung cancer, and leukemia but also breast cancer, ovary cancer, brain tumor, soft tissue tumour, and kidney tumor.

According to a fourth preferred aspect of the invention is disclosed a probe comprising any of DNA, DNA fragment, DNA oligomer, DNA primer, RNA, RNA fragment, RNA oligomer of a first group of embryonic stem cell genes with high level of expression in a cancer tumor and of a second group of embryonic stem cell genes with a low level of expression in said cancer tumor but not comprising DNA, DNA fragment, DNA oligomer, DNA primer, RNA, RNA fragment, RNA oligomer, respectively, of embryonic stem cell genes with an intermediate level of expression in said cancer tumor. It is preferred for the genes in the first and second groups to be those ranked, preferably consecutively, according to their expression levels by a one class significant analysis of microarrays (SAM) on a centered embryonic tumor stem cell gene dataset by employing a q value of from 0.01 to 0.1, more preferred of from 0.025 to 0.075, most preferred of about 0.05. The cancer can be any cancer, in particular prostate cancer, gastric cancer, lung cancer, and leukemia but also breast cancer, ovary cancer, brain tumor, soft tissue tumor, and kidney cancer.

According to a fifth preferred aspect of the invention is disclosed the use of a multitude of embryonic stem cell genes in a method of assessing the prognosis of a cancer tumor, wherein said multitude comprises a first group of embryonic stem cell genes with high level of expression in the tumor and of a second group of embryonic stem cell genes with a low level of expression in the tumor but does not comprise embryonic stem cell genes with an intermediate level of expression. It is preferred for the genes in the first and second groups to be ranked consecutively according to their expression levels and to constitute a fraction of the embryonic stem cell genes expressed in the tumor, in particular a fraction of 20 per cent or less of the embryonic stem cell genes expressed in the tumor. It is furthermore preferred to identify the multitude by a one class significant analysis of microarrays (SAM) on a centered embryonic tumor stem cell gene dataset by employing a q value of from 0.01 to 0.1, more preferred of from 0.025 to 0.075, most preferred of about 0.05. The use relates to any type of cancer, preferably prostate cancer, gastric cancer, lung cancer, and leukemia but also breast cancer, ovary cancer, brain tumor, soft tissue tumor, and kidney cancer.

According to a sixth preferred aspect of the invention the ESTP genes in the first group and the second group can be for analysis of clinical tumor tissue biopsies or tumor cell aspirate samples using high throughput DNA microarrays for clinical diagnosis and prognosis.

In a first preferred use is designed a gene microarray for probing the 641 or, less preferred, the aforementioned 1,000 or from 500 to 750 or, in particular, from 600 to 680 ESTP genes by spotting a DNA fragment (PCR products or oligos) of each of them on a glass or other suitable support. RNA isolated from tumor tissue biopsies or tumor cell aspirates can be labelled and hybridized with the ESTP gene microarray. The expression changes of all the 641 ES genes can be determined and compared with a group of standard reference cases with well defined data of clinical parameters such as histology, pathology and outcomes. The clinical outcomes of the new cases can thus be predicted.

A second preferred use relies on a gene solution array, for instance one based on the xMAP technology (http://www.luminexcorp.com). Probes that specifically bind to RNA of the ESTP genes can be designed, synthesized and immobilized on the surface of a microsphere or microbead support. RNA isolated from clinical tumor tissue biopsies or tumor cell aspirates can be bound to the support. Upon illuminating the beads/spheres with light of varying wavelength under laser beam activation the expression levels of the various ESTP genes in the tumor samples can be simultaneously and accurately measured. This method is simple, sensitive, and accurate and of high throughput; the expression levels of up to 100 genes can be in one experiment.

A third preferred use comprises the design of probes for assembling an ESTP gene microarray or chip of any kinds, for the purpose of application in clinical diagnosis and prognosis of common cancers.

According to a seventh preferred aspect of the invention high throughput PT-PCR can be used for analysis of clinical tumor tissue biopsies or tumor cell aspirate samples. Based on the ESTP gene list, design primers for each gene can be designed to carry out multiplex RT-PCR for determining the expression level of each gene in a tumor tissue or aspirate sample. Since the common RT-PCR platform can analyze 96 or multiple sets of 96 samples simultaneously, a small number of multiplex RT-PCR suffice to achieve high throughput measurement of the expression levels of the most preferred 641 ESTP genes or the less preferred 1000 or from 500 to 750 or, in particular, from 600 to 680 ESTP genes in a large set of clinical tumor tissue biopsies or aspirates.

According to an eight preferred aspect of the invention clinical tumor tissue biopsy samples and tumor cell aspirate samples can be analyzed using high throughput protein/antibody microarrays or an ELISA method. Based on the most preferred 641 ESTP genes or the less preferred 1000 or from 500 to 750 or, in particular, from 600 to 680 ESTP genes, the protein sequence or a portion thereof can be retrieved from publicly available human genome sequence resources and used to produce specific monoclonal antibodies for targeting the proteins encoded by the respective ESTP genes. The specific antibodies can be assembled into an ES protein array or incorporated into a high throughput ELISA system to measure the protein expression levels of the most preferred 641 ESTP genes and the less preferred 1000 or from 500 to 750 or, in particular, from 600 to 680 ESTP genes in clinical tumor tissue biopsies and tumor cell aspirates.

The invention will now be explained in greater detail by reference to preferred embodiments illustrated in a drawing.

### DESCRIPTION OF THE FIGURES

FIG. 1 is a graph illustrating the identification of ES predictor genes by a one-class SAM ranking test;

FIG. 2 is a gene expression profile obtained from biopsies of healthy and cancerous prostate tissue, and from embryonic stem cell lines, with a hierarchial clustering of the biopsies;

FIG. 3 is a gene expression profile obtained from biopsies of healthy and cancerous lung tissue biopsies, and from embryonic stem cell lines, with a hierarchial clustering of the biopsies;

FIG. 4 is a graph illustrating survival for the patients related to major cancerous lung tissue clusters of FIG. 3;

FIG. 5 is a gene expression profile obtained from biopsies of healthy and cancerous stomach tissue biopsies, and from embryonic stem cell lines, with a hierarchial clustering of the biopsies;

FIG. 6 is a graph illustrating survival for the patients related to major cancerous gastric tissue clusters of FIG. 5;

FIG. 7 is a gene expression profile obtained from leukocytes of acute myeloid leukemia patients, and from embryonic stem cell lines, with a hierarchial clustering of the leukocyte samples;

FIG. 8 is a graph illustrating survival for the patients pertaining to the major acute myeloid leukemia subtype clusters of FIG. 7.

### DESCRIPTION OF PREFERRED EMBODIMENTS

### Example 1

Data Retrieval. The method of the invention is based on published gene data such as the data sets published and deposited in the Stanford Microarray Database (SMD) (http://genome-www5.stanford.edu/). All array experiments used the same two-dye cDNA array platform with a common RNA reference, which enables reliable combination of or comparison with data from different experiments. These datasets include genome-wide expression data for embryonic stem cells (60), normal tissues from most of the human organs (61), and tumors from the prostate (62), breast, lung (63), stomach (64), liver (65), blood (66), brain (67), kidney (68), soft tissue (69), ovary (70; 71) and pancreas (72). In total about 1000 arrays were included in the analysis. Each array (tissue) in these datasets is denoted with corresponding basic clinical and pathological information such as histopathological type, tumor grade, clinical stage, and even survival data in a significant fraction of tumor cases.

Gene Selection. All genes or clones on arrays are selected. Control spots and empty spots are not included.

Data Collapse/Retrieval. Raw data are retrieved and averaged by SUID; UID column contains NAME; Retrieved Log(base2) of R/G Normalized Ratio (Mean). Data filtering options: Selected Data Filters: Spot is not flagged by experimenter. Data filters for GENEPIX result sets: Channel 1 Mean Intensity/Median Background Intensity>1.5 AND Channel 2 Normalized (Mean Intensity/Median Background Intensity)>1.5.

Data centering. The ES cell data set was combined with each of a number of other data sets. Genes and array batches were centered separately in each combined dataset as previously described (61; 62).

### Example 2

Identification of ES predictor genes. After centering a data set containing ES cells and normal tissues from most human organs, the ES data set was separated from the normal tissue data set. A one-class SAM (significant analysis of microarrays) was carried out using the centered ES dataset, by which all genes were ranked according to their expression levels in the ES cells (73). Using a q value equal to or less than 0.05 as cut-off, top 328 genes with highest level and top 313 genes with lowest level of expression in the ES cells were identified (Table 1). These 641 ES genes are named ES tumor predictor genes (ESTP genes). Previous studies used a small number of sample matrices to normalize the expression data of ES cells (60; 74); this may lead to erroneous identification of ESTP genes. In this invention, the expression data of ES genes from ES cells were centered by a matrix of over 100 normal tissues from most human organs (62). This greatly reduced erroneous identification of ESTP genes.

### Example 3

Prediction of clinical and pathological tumor types. After centering each combined data set, a sub-dataset containing only the 641 ESTP genes was isolated from the original dataset. A simple hierarchical clustering was carried out based on this sub-dataset using genes with 70% qualified data in all samples (78). The sample grouping was directly correlated with the clinical and pathological information of each individual tissue sample. Prediction examples for a number of tumor types are given below. Prediction in other datasets is carried out in essentially the same manner.

In the one class SAM analysis, numbers of genes selected is in correlation with q value. There were 201 genes selected when q value at 0.01, 641 genes selected when q value at 0.05, and 1368 genes selected when q value at 0.1. In other words, an increased q value would result in increased number of selected genes as well as increased number of genes that would not be associated with the transcriptional regulation in the ES cells.

Importantly, when the prediction powers were compared, the 641 genes selected by q value at 0.05 had best classification (prediction) results, as shown in the prostate cancer (Table 2) and lung cancer (Table 3) materials. The difference was particularly obvious in respect of lung cancer (Table 3). Thus the 641 genes selected by q value at 0.05 was the best choice of gene selection when both stem cell association and tumor classification are taken into consideration.

Definition of prediction. As described above, the ESTP genes were derived from the ES cell dataset. The power of this set of genes in the classification of a broad spectrum of tumors was then validated in each independent tumor dataset.

### Example 4

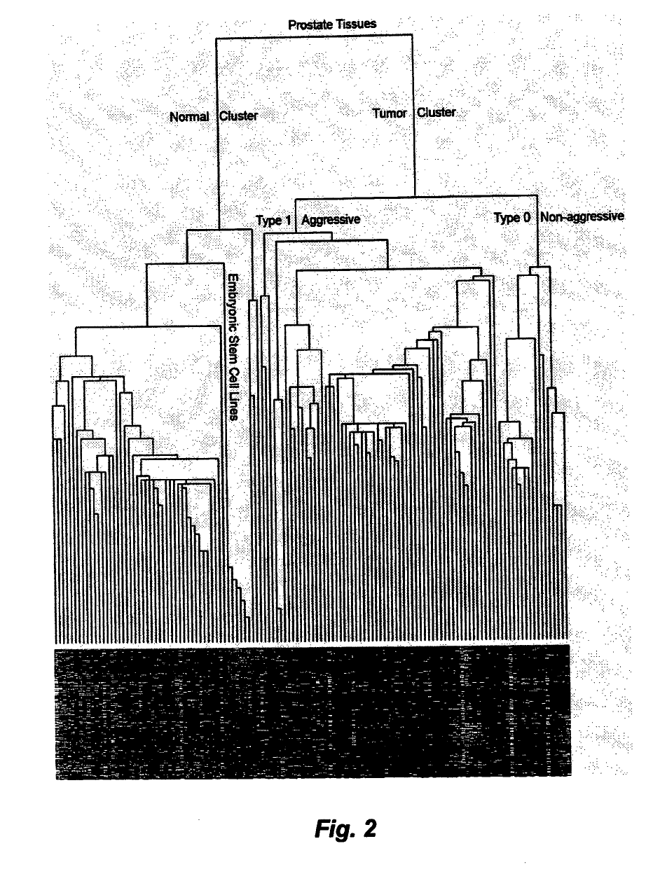

Prostate cancer. Published clinical data and predicted tumor subtype by ESTP genes of the invention for prostate cancer are listed in Table 2: Gleason grade, stage, biological subtype and short term recurrence (prostate specific antigen (PSA) survival) after radical surgery. Of the 641 ESTP genes, 505 had good data in 70% of all samples. In the gene expression profile of FIG. 2, the expression level (range in log ratio between −5.06 and 6.15) was transformed into a transitional color presentation, with red indicating above 0, black equal to 0 and green for less than 0; in FIG. 2 and the other figures illustrating gene expression profiles the colors are rendered in white, black, and grey (see, DESCRIPTION OF THE FIGURES). Based on these expression data, all samples were classified by hierarchical clustering into distinct groups as normal prostate, embryonic stem (ES) cells, prostate cancer group that contained all cases (66) with recurrence (PCa recurrent), Prostate cancer group that contained only cases without recurrence (PCa non-recurrent), and ES carcinoma cells. The classification is significantly (Fisher's exact test, p=0.001) correlated with the previous classification by using 5000 genes (Lapointe J et al., 2004). It should be noted that the PCa non-recurrent group predicted by the present invention is also significantly correlated with low Gleason score<6 (Fisher's exact test, p=0.028) and early stage (T<T3) (Fisher's exact test, p=0.007).

Prediction value for choice of treatment. Patients with a tumor predicted to be of a recurrent type (pertaining to the recurrent group) should be treated by radical surgery at a very early stage even in case of a moderate or low Gleason score. Patients with a very early stage tumor predicted to be of a non-recurrent type (pertaining to the non-recurrent group) should be kept under regular PSA and other examination control, because most of the tumors in this group are in fact indolent or very slow-progressive.

### Example 5

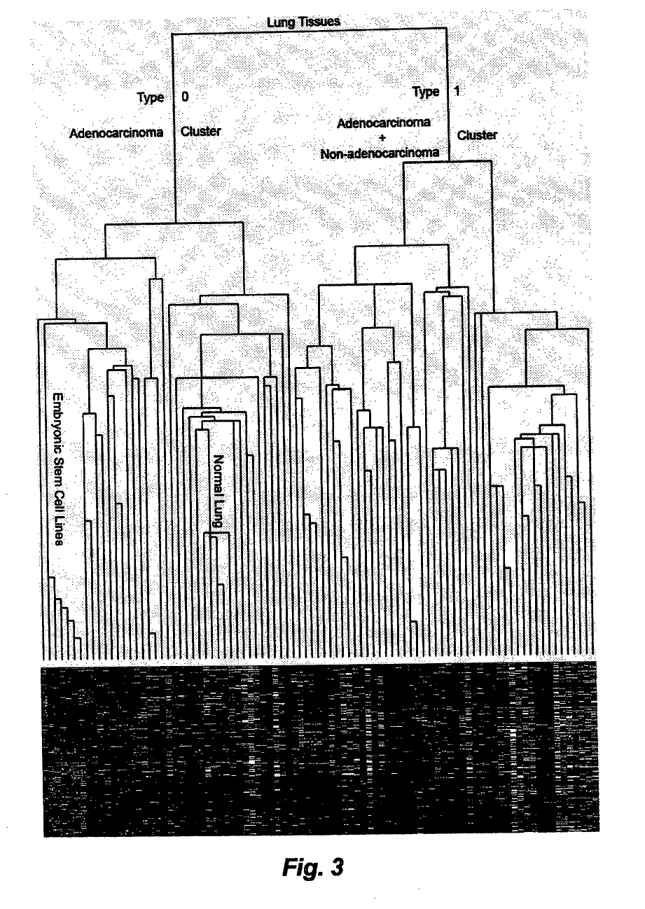

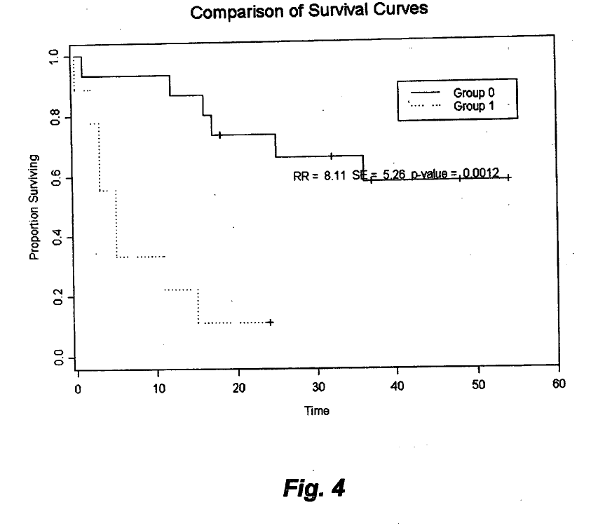

Lung cancer. Published clinical data and predicted tumor subtype by ESTP genes of the invention are shown in Table 3. Prediction of histological type and survival in lung cancer is illustrated in FIG. 3, tissue clustering by ESTP genes. Of the 641 ES predictor genes, 316 had qualified data in 70% or more of the samples. Lung cancer tissue samples were predictively sorted into two major groups, an adenocarcinoma group (a) that mainly contained adenocarcinomas, some normal lung tissues, ES cells and a few non-adenocarcinomas, and a (b) non-adenocarcinoma group that contained most non-adenocarcinomas including squamous cell carcinoma, large cell lung cancer and small cell lung cancer, together with a fraction of adenocarcinomas. In general, adenocarcinoma has a better prognosis than other types of lung cancer. Survival analysis based on lung adenocarcinoma subtypes is illustrated in FIG. 4.

The adenocarcinoma cases in the non-adenocarcinoma group (b) further showed shorter survival than adenocarcinoma cases in the adenocarcinoma group (a) as shown in FIG. 3, adenocarcinoma subtypes by ES predictor genes associated with survival.

Predictive value for choice of treatment strategy: tumors predicted to pertain to the adenocarcinoma group seem to have a generally favorable outcome after radical surgery at a very early stage; whereas tumors in the non-adenocarcinoma group may respond relatively better to chemotherapy such as to Iressa or radiation.

### Example 6

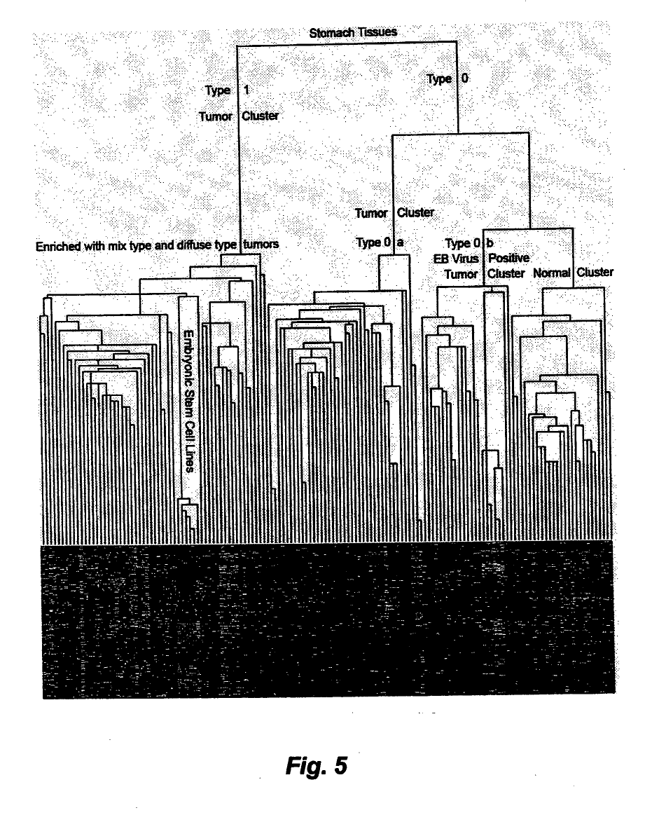

Gastric cancer. Published clinical data and tumor subtype predicted by ESTP genes of the invention are illustrated in Table 4. The prediction of histological types and survival in gastric cancer is illustrated in FIG. 5: (a) tissue clustering by ES predictor genes; (b) issue subtypes by ES predictor genes associated with survival.

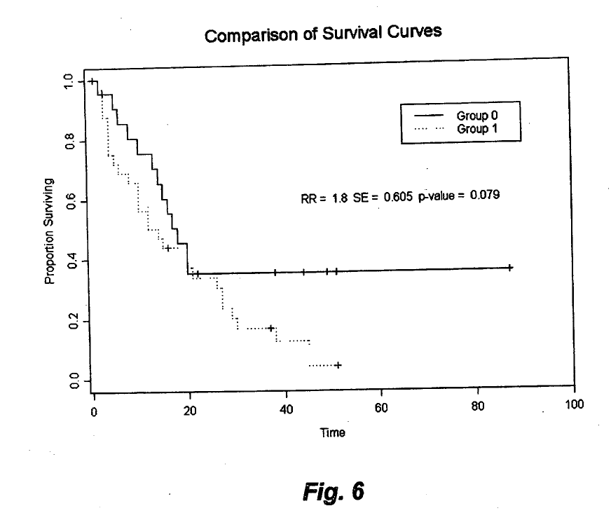

Prediction of subtypes of gastric cancer by ESTP genes: of the 641 ESTP genes 613 had qualified data in 70% of all samples. Gastric tumors were classified into two major subtypes, type 1 enriched in tumors with diffuse and mix histological types generally with poor prognosis, type 0 together with most normal gastric tissue samples. The survival time for gastric cancer patients pertaining to these groups is compared in FIG. 6. The subtype 0 tumors can be further divided into two sub-subtypes, one with the A subtype enriched in EB virus positive tumors, the other not.

Predictive value: a) EBV infection is linked to gastric cancer via stem cell biology. Preventing an EBV infection by vaccination may have preventive effect on gastric cancer; b) Diffused type of gastric cancer has very strong hereditary tendency. One should specifically exclude gastric cancer in a relative to a patient whose tumor is predicted to pertain to this group, so that possible tumor can be treated radically at a very early stage.

### Example 7

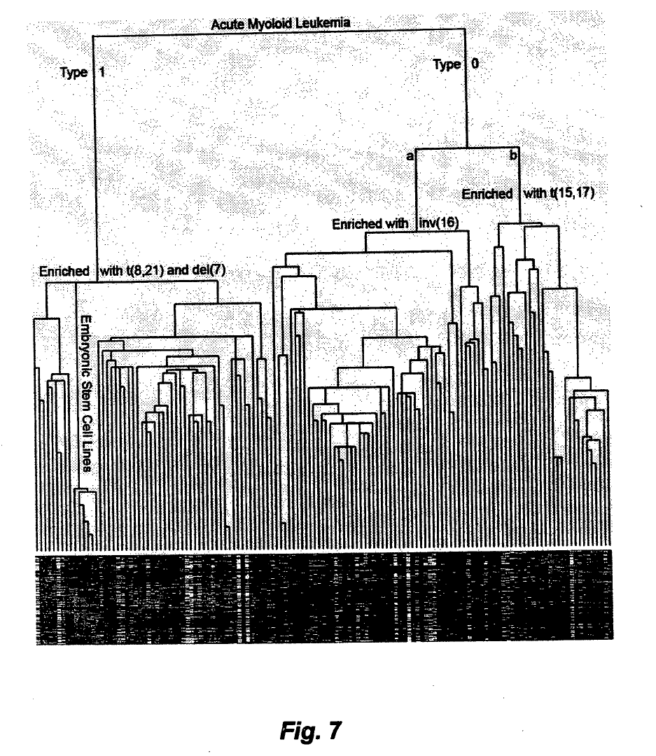

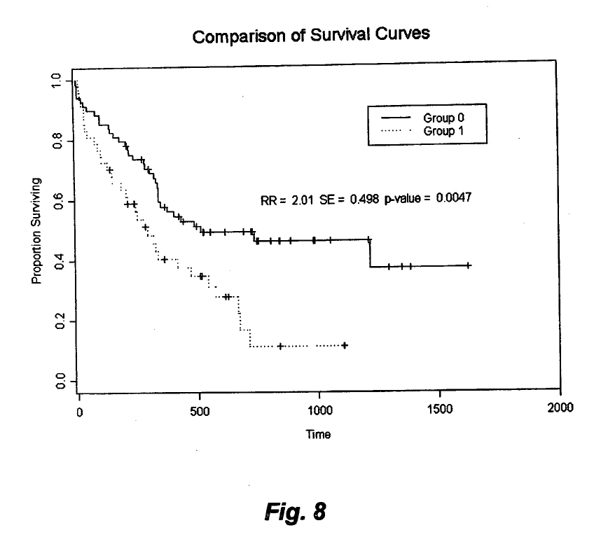

Leukemia. Published clinical data and predicted tumor subtype by ESTP genes of the invention are listed in Table 5. FIG. 7 illustrates the prediction of subtypes of acute mononucleocyte leukemia associated with chromosome aberration and survival: (a) classification by ESTP genes; (b) AML subtypes associated with survival. Prediction of acute myeloid leukemia (AML) by ESTP genes: of the 641 ES predictor genes, 324 had qualified data in 70% of all samples. AML cases were classified into two major subtypes, type 1 enriched in cases with t(8;21) and del7q chromosomal aberrations, and type 0, which was further divided into two sub-subtypes A and B the first with a subtype enriched with inv(16), the second enriched with t(15;17). Type 1 cases showed shorter overall survival than type 0 as presented in FIG. 8. Survival analysis was based on AML subtypes predicted in FIG. 4a and the published clinical data in Table 5.

Predictive value for treatment choices: AML with different chromosomal aberrations responds to different chemotherapies; in particular all-trans retinoic acid can induce differentiation of AML with t(15;17) translocation. It is suggested that AML in the group enriched with t(15;17) but without the translocation detected by cytogenetic diagnostic method may show good response to all-trans retinoic acid due to the same stem cell biological alteration.

### Example 8

Case History and Retrospective Cancer Treatment Strategy Suggested by the Method of the Invention.

(a) Prostate cancer patient #PC007 (Table 5) aged 56 y at diagnosis. Gleason score of prostate cancer was 3+3=6; tumor stage was T2b, suggesting a well differentiated tumor at an early stage by conventional clinical pathological examination. In spite of this the tumor recurred as diagnosed by a re-increased PSA level 27.7 months after radical surgery. According to the predictive method of the invention, the tumor is predicted to be of ES type 1 with poor prognosis. This case illustrates a typical situation in which ES type prediction can outperform conventional clinical pathological methods in predicting clinical outcome. A similar case is patient PC250 (Table 5).

(b) Prostate cancer patient #PC037 (Table 5). This 57 year-old patient had a Gleason 4+3 tumor, a high grade tumor that would have a poor prognosis according to conventional clinical concepts. But, according to the predictive method of the invention, the tumor is classified as being of ES type 0 and thus would have had a better prognosis. The patient had a radical surgery without any signs of recurrence after 16.2 months. This case provides also an example for the situation that the ES typing in the present invention is superior to conventional Gleason grading.

(c) Prostate cancer patient #PC092 (Table 5). This patient was aged 68 y at diagnosis. His tumor had Gleason 3+3=6 and staged T2b, suggesting a well differentiated tumor at an early stage. By the method of the present invention the tumor is classified as being of ES type 0 with good prognosis. The patient was treated by radical surgery. No signs of recurrence were observed 13.7 months post surgery. There is good agreement between Gleason grading and ES typing according to the present invention. The ES typing result also suggests that the patient could have been safely kept under regular PSA control instead of immediate radical surgery.

### Example 9

Prognosis of lung adenocarcinoma. In addition to the prostate cancer cases from Table 5 elucidated above, it is seen that ES typing according to the present invention is significantly better than conventional histological grading in the prognosis of lung adenocarcinoma. For example, cases #222-97 and #226-97 were of grade 3 that would be poorly differentiated with poor outcome according to conventional clinical prognostic methods. By the method of the present invention the cases are classified as being of ES type 0 that would have a relatively good outcome. The patients were recurrence-free more than 48 months after radical surgery. Again ES typing by the method of the invention is more accurate than by conventional histological grading.

### Legends to Figures

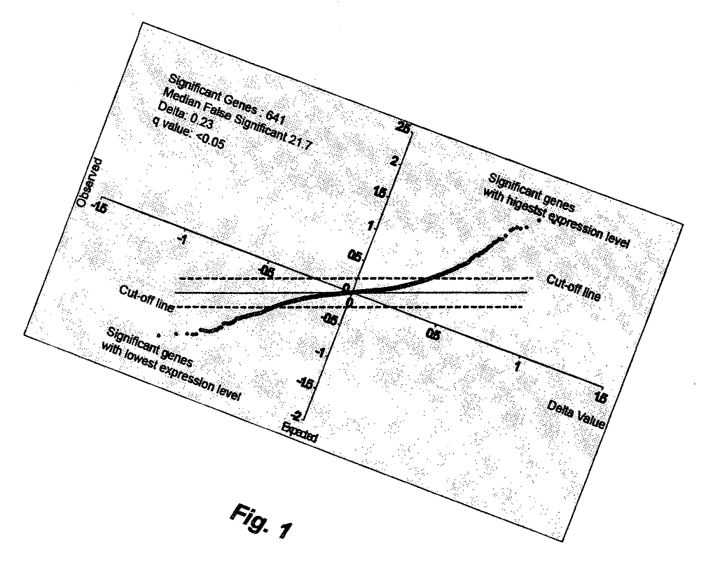

FIG. 1. Identification of ESTP genes by a one-class SAM ranking test. There were 24361 genes with qualified expression data in 75% of the 6 embryonic stem (ES) cell lines. These 24361 genes were ranked according to their homogenous expression levels in the ES cells by a one-class SAM (significant analysis of microarrays) method as shown in this figure. At delta 0.23, q value<0.05, 328 genes with highest expression levels and 313 genes with lowest expression levels were identified. The expression changes of these 641 genes in different tumor samples showed also strongest classification power as compared to genes located within the cut-off lines. Increasing the delta value (decreasing the q value) can increase the specificity in selecting genes representing the transcriptional regulation in the ES cells whereas it can decrease the number of selected genes. A decrease in significant genes selected could result in a decrease in the corresponding tumor classification power. By successively changing the cut-off line it was shown that the 641 genes selected at delta 0.23, q value<0.05 was the best choice for both stem cell association and tumor classification.

FIG. 2. Prediction of prostate cancer—Gleason grade, stage, biological subtype and short term recurrence (prostate specific antigen (PSA) survival) after radical surgery. Of the 641 ESTP genes, 505 had good data in 70% of all samples. In this gene expression profile, the expression level (range in log ratio between −5.06 and 6.15) was transformed into a transitional gray-black scale presentation, with black indicating above 1, median gray indicate equal to 1 and green for less than 1. Based on these expression data, all samples were classified by hierarchical clustering into distinct groups as normal prostate, prostate cancer aggressive group type 1 that contained all cases with recurrence, prostate cancer non-aggressive group type 0 that contained only cases without recurrence. The classification significantly (Fisher's exact test, p=0.001) correlated with the previous classification by using 5000 genes (Lapointe J et al., 2004). The non-aggressive group predicted by the present invention was also significantly correlated with low Gleason score <6 (Fisher's exact test, p=0.028) and early stage (T<T3) (Fisher's exact test, p=0.007).

One tumor sample was provided for each prostate cancer patient. For some prostate cancer patients also a healthy (“normal”) tissue sample was provided from an unaffected prostate area. These normal samples formed the “normal” cluster in FIG. 1. There were 6 embryonic stem (ES) cell lines from non-prostate cancer subjects. In addition 10 embryonic carcinoma (EC) cell lines from patients with embryonic carcinoma were included. These ES and EC cell lines were used as reference to illustrate different patterns of gene expression. Importance of this prediction for treatment choices: patients whose tumor is predicted in the aggressive group type 1 should be treated by radical surgery at very early stage even if the tumor Gleason score is not high; whereas patients whose tumor is predicted in the non-aggressive group type 0 should be under regular PSA and other examination control if the tumor is at very early stage, because most of the tumors in this group are in fact indolent or progress very slowly.

FIG. 3. Prediction of lung cancer tissue type. Of the 641 ESTP genes, 316 had qualified data in 70% or more of the samples. Lung cancer tissue samples were predicted into two major groups, adenocarcinoma group type 0 that mainly contained adenocarcinomas, some normal lung tissues, ES cells and a few non-adenocarcinomas, and non-adenocarcinoma group type 1 that contained most non-adenocarcinomas including squamous cell carcinoma, large cell lung cancer and small cell lung cancer, together with a fraction of adenocarcinomas. In general, adenocarcinoma has relatively better prognosis than other types of lung cancer. In this invention, the adenocarcinoma cases in the non-adenocarcinoma group type 1 further showed shorter survival than adenocarcinoma cases in the adenocarcinoma group type 0 as shown in FIG. 4.

All lung cancer patients had a tumor sample. A few patients had also a normal sample from the unaffected lung areas. These a few normal samples clustered together as shown in this figure. There were 6 embryonic stem (ES) cell lines from non-prostate cancer subjects. In addition 10 embryonic carcinoma (EC) cell lines from patients with embryonic carcinoma were also included. These ES and EC cell lines were used as reference to indicate different patterns of gene expression.

Importance of the prediction for treatment strategy: tumors predicted in the adenocarcinoma group may have favourable outcome after radical surgery at very early stage.

FIG. 4. Lund adenocarcinoma survival analysis. The analysis is based on lung adenocarcinoma subtypes predicted in FIG. 3 and the published clinical data reproduced in Table 3. Time unit: months.

FIG. 5. Prediction of subtypes of gastric cancer by ESTP genes. Of the 641 ESTP genes, 613 had qualified measuring in 70% of all samples. Gastric tumors were classified into two major subtypes, type 1 enriched with diffuse type and mix type tumors generally with poor prognosis, type 0 together with most normal gastric tissue samples. Type 0 tumors was further divided into two subtypes with the a subtype enriched with tumors with EB virus-positive.

One tumor sample was provided from each gastric cancer patient. From some of the patients also a normal sample was taken from an unaffected stomach area. These “normal” samples formed the normal cluster in FIG. 5. There were 6 embryonic stem (ES) cell lines from non-prostate cancer subjects. In addition 10 embryonic carcinoma (EC) cell lines from patients with embryonic carcinoma were also included. These ES and EC cell lines were used as reference to indicate different patterns of gene expression.

Importance of the prediction: a) EBV infection is linked to gastric cancer via stem cell biology. Preventing EBV infection by vaccination may have preventing effect on gastric cancer; b) diffused type of gastric cancer has a very strong hereditary tendency. One should specifically exclude gastric cancer in a relative to a patient, whose tumor is predicted in this group, so that a tumor, if detected, can be treated radically at very early stage.

FIG. 6. Gastric cancer survival analysis. The analysis was based on gastric cancer subtypes predicted in FIG. 5 and on the published clinical data reproduced in Table 4. Time unit: months.

FIG. 7. Prediction of acute myeloid leukemia (AML) by ESTP genes. Of the 641 ES predictor genes, 324 had qualified data in 70% of all samples. AML cases were classified into two major subtypes, type 1 enriched in cases with t(8;21) and del7q chromosomal aberrations, type 0 that was further divided into two subtypes a and b with a subtype enriched inv(16) and b subtype enriched with t(15;17). Type 1 cases showed shorter overall survival than type 0 as presented in FIG. 5.

From each patient one leukocyte sample was harvested. There were 6 embryonic stem (ES) cell lines from non-prostate cancer subjects. In addition 10 embryonic carcinoma (EC) cell lines from patients with embryonic carcinoma were also included. These ES and EC cell lines were used as reference to indicate different patterns of gene expression.

Importance of the prediction for treatment choices: AML with different chromosomal aberrations respond to different chemotherapies, in particular all-trans retinoic acid can induce differentiation of AML with t(15;17) translocation. It is highly possible that AML in the group enriched with t(15;17) but without the translocation detected by cytogenetic diagnostic method can show good response to all-trans retinoic acid due to the same stem cell biological alteration.

FIG. 8. Leukemia survival analysis. The analysis was based on AML subtypes predicted in FIG. 7 and on the published clinical data reproduced in Table 5. Time unit: months.

### REFERENCES

1. Lapointe J et al., Gene expression profiling identifies clinically relevant subtypes of prostate cancer. Proc Natl Acad Sci USA, 2004; 101(3): 811-816.
2. Perou C M, et al., Molecular portraits of human breast tumours. Nature, 2000; 406(6797): 747-752.
3. Singh R et al., Microarray based comparison of three amplification methods for nanogram amounts of total RNA. Am J Physiol Cell Physiol, 2004.
4. Sorlie T et al., Gene expression patterns of breast carcinomas distinguish tumor subclasses with clinical implications. Proc Natl Acad Sci USA, 2001; 98(19): 10869-10874.
5. van de Vijver M J et al., A gene-expression signature as a predictor of survival in breast cancer. N Engl J Med, 2002; 347(25): 1999-2009.
6. van 't Veer L J et al., Gene expression profiling predicts clinical outcome of breast cancer. Nature, 2002; 415(6871): 530-536.
7. Varambally S et al., The polycomb group protein EZH2 is involved in progression of prostate cancer. Nature 2002; 419(6907): 624-629.
8. Eisen M B et al., Cluster analysis and display of genome-wide expression patterns. Proc Natl Acad Sci USA, 1998; 95(25): 14863-14868.
9. Tusher V G et al., Significance analysis of microarrays applied to the ionizing radiation response. Proc Natl Acad Sci USA, 2001; 98(9): 5116-5121.
10. Sherlock G, Of fish and chips. Nat Methods, 2005; 2(5): 329-330.
11. Isaacs W et al., Focus on prostate cancer. Cancer Cell, 2002; 2(2): 113-116.
12. Jemal A et al., Cancer Statistics, 2005. CA Cancer J Clin, 2005; 55(1): 10-30.
13. Holmberg L et al., A randomized trial comparing radical prostatectomy with watchful waiting in early prostate cancer. N Engl J Med, 2002; 347(11): 781-789.
14. Johansson J E et al., Natural history of early, localized prostate cancer. Jama, 2004; 291(22): 2713-2719.
15. Humphrey P A, Gleason grading and prognostic factors in carcinoma of the prostate. Mod Pathol, 2004; 17(3): 292-306.
16. Gleason D F and Mellinger G T, Prediction of prognosis for prostatic adenocarcinoma by combined histological grading and clinical staging. J Urol, 1974; 111(1): 58-64.
17. Partin A W et al., Combination of prostate-specific antigen, clinical stage, and Gleason score to predict pathological stage of localized prostate cancer. A multi-institutional update. Jama, 1997; 277(18): 1445-1451.
18. Partin A W et al., The use of prostate specific antigen, clinical stage and Gleason score to predict pathological stage in men with localized prostate cancer. J Urol, 1993; 150(1): 110-114.
19. Tricoli J V et al., Detection of prostate cancer and predicting progression: current and future diagnostic markers. Clin Cancer Res, 2004; 10(12 Pt 1): 3943-3953.
20. Cahill D P et al., Genetic instability and darwinian selection in tumours. Trends Cell Biol, 1999; 9(12): M57-60.
21. Hahn W C et al., Creation of human tumour cells with defined genetic elements. Nature, 1999; 400(6743): 464-468.
22. Hahn W C and Weinberg R A, Rules for making human tumor cells. N Engl J Med, 2002; 347(20): 1593-1603.
23. Hahn W C and Weinberg R A, Modeling the molecular circuitry of cancer. Nat Rev Cancer, 2002; 2(5): 331-341.
24. Lengauer C et al., Genetic instabilities in human cancers. Nature, 1998; 396(6712): 643-649.
25. Vogelstein B and Kinzler K W, The multistep nature of cancer. Trends Genet, 1993; 9(4): 138-141.
26. Vogelstein B and Kinzler K W, Cancer genes and the pathways they control. Nat Med, 2004; 10(8): 789-799.
27. Cairns P et al., Frequent inactivation of PTEN/MMAC1 in primary prostate cancer. Cancer Res, 1997; 57(22): 4997-5000.
28. Carpten J et al., Germline mutations in the ribonuclease L gene in families showing linkage with HPC1. Nat Genet, 2002; 30(2): 181-184.
29. Huusko P et al., Nonsense-mediated decay microarray analysis identifies mutations of EPHB2 in human prostate cancer. Nat Genet, 2004; 36(9): 979-983.
30. Li J et al., PTEN, a putative protein tyrosine phosphatase gene mutated in human brain, breast, and prostate cancer. Science, 1997; 275(5308): 1943-1947.
31. Steck P A et al., Identification of a candidate tumour suppressor gene, MMAC1, at chromosome 10q23.3 that is mutated in multiple advanced cancers. Nat Genet, 1997; 15(4): 356-362.
32. Taplin M E et al., Mutation of the androgen-receptor gene in metastatic androgen-independent prostate cancer. N Engl J Med, 1995; 332(21): 1393-1398.
33. Tavtigian S V et al., A candidate prostate cancer susceptibility gene at chromosome 17p. Nat Genet, 2001; 27(2): 172-180.
34. Visakorpi T et al., In vivo amplification of the androgen receptor gene and progression of human prostate cancer. Nat Genet, 1995; 9(4): 401-406.
35. De Marzo A M et al., Human prostate cancer precursors and pathobiology. Urology, 2003; 62(5 Suppl 1): 55-62.
36. Nelson W G et al., Prostate cancer. N Engl J Med, 2003; 349(4): 366-381.
37. Schena M, Shalon D, Davis R W, and Brown P O Quantitative monitoring of gene expression patterns with a complementary DNA microarray. Science, 1995; 270(5235): 467-470.
38. Bettuzzi S et al., Successful prediction of prostate cancer recurrence by gene profiling in combination with clinical data: a 5-year follow-up study. Cancer Res, 2003; 63(13): 3469-3472.
39. Bueno R et al., A diagnostic test for prostate cancer from gene expression profiling data. J Urol, 2004; 171(2 Pt 1): 903-906.
40. Chetcuti A et al., Identification of differentially expressed genes in organ-confined prostate cancer by gene expression array. Prostate, 2001; 47(2): 132-140.
41. Dhanasekaran S M et al., Delineation of prognostic biomarkers in prostate cancer. Nature, 2001; 412(6849): 822-826.
42. Elek J et al., Microarray-based expression profiling in prostate tumors. In Vivo, 2000; 14(1): 173-182.
43. Febbo P G and Sellers W R, Use of expression analysis to predict outcome after radical prostatectomy. J Urol, 2003; 170(6 Pt 2): S11-19; discussion S19-20.
44. Glinsky G V et al., Gene expression profiling predicts clinical outcome of prostate cancer. J Clin Invest, 2004; 113(6): 913-923.
45. Henshall S M et al., Survival analysis of genome-wide gene expression profiles of prostate cancers identifies new prognostic targets of disease relapse. Cancer Res, 2003; 63(14): 4196-4203.
46. Latil A et al., Gene expression profiling in clinically localized prostate cancer: a four-gene expression model predicts clinical behavior. Clin Cancer Res, 2003; 9(15): 5477-5485.
47. LaTulippe E et al., Comprehensive gene expression analysis of prostate cancer reveals distinct transcriptional programs associated with metastatic disease. Cancer Res, 2002; 62(15): 4499-4506.
48. Luo J et al., Human prostate cancer and benign prostatic hyperplasia: molecular dissection by gene expression profiling. Cancer Res, 2001; 61(12): 4683-4688.
49. Luo J et al., Gene expression signature of benign prostatic hyperplasia revealed by cDNA microarray analysis. Prostate, 2002; 51(3): 189-200.
50. Magee J A et al., Expression profiling reveals hepsin overexpression in prostate cancer. Cancer Res, 2001; 61(15): 5692-5696.
51. Nelson P S, Predicting prostate cancer behavior using transcript profiles. J Urol, 2004; 172(5 Pt 2): S28-32; discussion S33.
52. Singh D et al., Gene expression correlates of clinical prostate cancer behavior. Cancer Cell, 2002; 1(2): 203-209.
53. Xu J et al., Identification of differentially expressed genes in human prostate cancer using subtraction and microarray. Cancer Res, 2000; 60(6): 1677-1682.
54. Yu Y P et al., Gene expression alterations in prostate cancer predicting tumor aggression and preceding development of malignancy. J Clin Oncol, 2004; 22(14): 2790-2799.
55. Andersson L et al., Fine needle aspiration biopsy for diagnosis and follow-up of prostate cancer. Consensus Conference on Diagnosis and Prognostic Parameters in Localized Prostate Cancer. Stockholm, Sweden, May 12-13, 1993. Scand J Urol Nephrol Suppl, 1994; 162(43-49; discussion 115-127.
56. Brolin J et al., Immunocytochemical detection of the androgen receptor in fine needle aspirates from benign and malignant human prostate. Cytopathology, 1992; 3(6): 351-357.
57. Assersohn L et al., The feasibility of using fine needle aspiration from primary breast cancers for cDNA microarray analyses. Clin Cancer Res, 2002; 8(3): 794-801.
58. Goley E M et al., Microarray analysis in clinical oncology: pre-clinical optimization using needle core biopsies from xenograft tumors. BMC Cancer, 2004; 4(1): 20.
59. Li Y et al., Direct comparison of microarray gene expression profiles between non-amplification and a modified cDNA amplification procedure applicable for needle biopsy tissues. Cancer Detect Prev, 2003; 27(5): 405-411.
60. Sperger J M et al., Gene expression patterns in human embryonic stem cells and human pluripotent germ cell tumors. Proc Natl Acad Sci USA, 2003; 100(23): 13350-13355.
61. Shyamsundar R et al., Correction: A DNA microarray survey of gene expression in normal human tissues. Genome Biol, 2005; 6(9): 404.
62. Lapointe J et al., Gene expression profiling identifies clinically relevant subtypes of prostate cancer. Proc Natl Acad Sci USA, 2004; 101(3): 811-816.
63. Garber M E et al., Diversity of gene expression in adenocarcinoma of the lung. Proc Natl Acad Sci USA, 2001; 98(24): 13784-13789.
64. Chen X et al., Variation in gene expression patterns in human gastric cancers. Mol Biol Cell, 2003; 14(8): 3208-3215.
65. Chen X et al., Gene expression patterns in human liver cancers. Mol Biol Cell, 2002; 13(6): 1929-1939.
66. Bullinger L et al., Use of gene-expression profiling to identify prognostic subclasses in adult acute myeloid leukemia. N Engl J Med, 2004; 350(16): 1605-1616.
67. Liang Y et al., Gene expression profiling reveals molecularly and clinically Distinct subtypes of glioblastoma multiforme. Proc Natl Acad Sci USA, 2005; 102(16): 5814-5819.
68. Higgins J P et al., Gene expression patterns in renal cell carcinoma assessed by complementary DNA microarray. Am J Pathol, 2003; 162(3): 925-932.
69. Nielsen T O et al., Molecular characterisation of soft tissue tumours: a gene expression study. Lancet, 2002; 59(9314): 1301-1307.
70. Schaner M E et al., Variation in gene expression patterns in effusions and primary tumors from serous ovarian cancer patients. Mol Cancer, 2005; 4(26).
71. Schaner M E et al., Gene expression patterns in ovarian carcinomas. Mol Biol Cell, 2003; 14(11): 4376-4386.
72. Iacobuzio-Donahue C A et al., Exploration of global gene expression patterns in pancreatic adenocarcinoma using cDNA microarrays. Am J Pathol, 2003; 162(4): 1151-1162.
73. Tusher V G et al., Significance analysis of microarrays applied to the ionizing gradiation response. Proc Natl Acad Sci USA, 2001; 98(9): 5116-5121.
74. Skottman H et al., Gene expression signatures of seven individual human embryonic stem cell lines. Stem Cells, 2005; 23(9): 1343-1356.
75. Shamir R et al., R EXPANDER—an integrative program suite for microarray data analysis. BMC Bioinformatics, 2005; 6(232).
76. Lee H K et al., Ermine J: tool for functional analysis of gene expression data sets. BMC Bioinformatics, 2005; 6(269).
77. Diehn M et al., Genome-Scale. Identification of Membrane-Associated Human mRNAs. PLoS Genet, 2006; 2(1): e11.
78. Eisen M B et al., Cluster analysis and display of genome-wide expression patterns. Proc Natl Acad Sci USA, 1998; 95(25): 14863-14868.

TABLE 1
Genes with extreme (highest and lowest) expression levels in ES cells

| Strongly positive expression level score (d) | | | | Strongly negative expression level score (d) | | | |
|---|---|---|---|---|---|---|---|
| (continued on the left of the following pages) | | | | (continued on the right of the following pages) | | | |
| IMAGE | Gene | | q-Value × | IMAGE | Gene | | q-Value × |
| clone | symbol | Score (d) | 10² | clone | symbol | Score (d) | 10² |
| 840944 | EGR1 | 2.00 | 0.67 | 490023 | WNT5B | −1.61 | 0.67 |
| 753104 | DCT | 1.95 | 0.67 | 433257 | LOC285458 | −1.49 | 0.67 |
| 1680098 | Hs.545599 | 1.79 | 0.67 | 1628121 | ABCG2 | −1.43 | 0.67 |
| 1944026 | TAGLN | 1.74 | 0.67 | 781289 | AA429944 | −1.41 | 0.67 |
| 898092 | CTGF | 1.74 | 0.67 | 796542 | ETV5 | −1.39 | 0.67 |
| 526657 | TCEB3 | 1.70 | 0.67 | 1948085 | GBR3 | −1.30 | 0.67 |
| 526184 | Hs.551490 | 1.67 | 0.67 | 2017535 | LRP4 | −1.29 | 0.67 |
| 384111 | AA702568 | 1.57 | 0.67 | 1556056 | PRPH | −1.29 | 0.67 |
| 452134 | AA707225 | 1.51 | 0.67 | 462144 | ARSE | −1.29 | 0.67 |
| 360254 | CYR61 | 1.49 | 0.67 | 415619 | SLC5A9 | −1.28 | 0.67 |
| 80186 | Hs.534427 | 1.49 | 0.67 | 1389018 | CA4 | −1.27 | 0.67 |
| 301068 | Hs.433075 | 1.44 | 0.67 | 143966 | SEPT6 | −1.25 | 0.67 |
| 1607286 | CYR61 | 1.42 | 0.67 | 502151 | SLC16A3 | −1.24 | 0.67 |
| 378488 | CYR61 | 1.42 | 0.67 | 1519951 | ETV5 | −1.22 | 0.67 |
| 306841 | Hs.419777 | 1.37 | 0.67 | 450938 | DKFZP586A0522 | −1.22 | 0.67 |
| 53245 | LOC150383 | 1.35 | 0.67 | 1323448 | CRIP1 | −1.19 | 0.67 |
| 1660645 | CYP26A1 | 1.32 | 0.67 | 324593 | MGC16291 | −1.17 | 0.67 |
| 33837 | FRAS1 | 1.29 | 0.67 | 824933 | NF1 | −1.16 | 0.67 |
| 2012523 | STX3A | 1.27 | 0.67 | 1742419 | WNT11 | −1.10 | 0.67 |
| 38642 | CYP26A1 | 1.26 | 0.67 | 70152 | DKFZP586A0522 | −1.09 | 0.67 |
| 1473274 | MYL9 | 1.23 | 0.67 | 1613496 | Hs.505172 | −1.08 | 0.67 |
| 1434897 | COL5A2 | 1.22 | 0.67 | 461488 | ARRB1 | −1.08 | 0.67 |
| 307244 | LIPL3 | 1.22 | 0.67 | 783697 | AA446838 | −1.07 | 0.67 |
| 1567658 | AA976207 | 1.21 | 0.67 | 22355 | RGS4 | −1.07 | 0.67 |
| 49707 | Hs.517502 | 1.20 | 0.67 | 913672 | Hs.430369 | −1.07 | 0.67 |
| 950676 | KIF1A | 1.17 | 0.67 | 1521792 | IBRDC3 | −1.07 | 0.67 |
| 843098 | BASP1 | 1.17 | 0.67 | 51672 | Hs.548513 | −1.06 | 0.67 |
| 129320 | FRAS1 | 1.17 | 0.67 | 76182 | CCDC3 | −1.06 | 0.67 |
| 43745 | SYT6 | 1.17 | 0.67 | 1554367 | TXNIP | −1.06 | 0.67 |
| 204335 | CD24 | 1.16 | 0.67 | 454459 | FBXL14 | −1.04 | 0.67 |
| 1946026 | FLJ10884 | 1.15 | 0.67 | 72003 | IL6R | −1.04 | 0.67 |
| 179534 | KCNQ2 | 1.15 | 0.67 | 429093 | LOC285458 | −1.03 | 0.67 |
| 898218 | IGFBP3 | 1.14 | 0.67 | 810303 | Hs.451488 | −1.02 | 0.67 |
| 782476 | GULP1 | 1.13 | 0.67 | 120162 | Hs.535086 | −1.01 | 0.67 |
| 309929 | GPR | 1.11 | 0.67 | 1324242 | TNFSF7 | −1.01 | 0.67 |
| 756372 | RARRES2 | 1.11 | 0.67 | 731255 | Hs.487536 | −1.00 | 0.67 |
| 1500247 | AA886761 | 1.09 | 0.67 | 32576 | CCDC3 | −0.97 | 0.67 |
| 281039 | FABP5 | 1.08 | 0.67 | 416408 | Hs.79856 | −0.96 | 0.67 |
| 79598 | CDH1 | 1.06 | 0.67 | 2009000 | GNB3 | −0.95 | 0.67 |
| 810728 | ZD52F10 | 1.04 | 0.67 | 379768 | CRLF1 | −0.95 | 0.67 |
| 1883559 | FST | 1.04 | 0.67 | 1473171 | TXNIP | −0.95 | 0.67 |
| 51807 | FHOD3 | 1.03 | 0.67 | 502656 | IMPA3 | −0.95 | 0.67 |
| 1607473 | Hs.157101 | 1.01 | 0.67 | 594758 | Hs.529095 | −0.95 | 0.67 |
| 66977 | AIG1 | 1.01 | 0.67 | 260170 | N32072 | −0.95 | 0.67 |
| 927112 | KIAA0773 | 1.00 | 0.67 | 2028002 | ABCD1 | −0.94 | 0.67 |
| 361974 | PTN | 1.00 | 0.67 | 32110 | ABCG2 | −0.93 | 0.81 |
| 880630 | MGC3036 | 0.99 | 0.67 | 781738 | GATA4 | −0.93 | 0.81 |
| 786609 | COL12A1 | 0.99 | 0.67 | 296140 | MGC15887 | −0.93 | 0.81 |
| 1607129 | POU5F1 | 0.99 | 0.67 | 1928791 | F3 | −0.93 | 0.81 |
| 210921 | NFKBIZ | 0.98 | 0.67 | 489594 | ZCWCC2 | −0.92 | 0.81 |
| 878850 | GCAT | 0.98 | 0.67 | 1257131 | Hs.552645 | −0.92 | 0.81 |
| 281100 | SYT6 | 0.98 | 0.67 | 243410 | GATA4 | −0.91 | 0.81 |
| 788234 | ID4 | 0.96 | 0.67 | 685489 | Hs.505172 | −0.91 | 0.81 |
| 774446 | ADM | 0.96 | 0.67 | 178825 | NRGN | −0.91 | 0.81 |
| 34140 | GCA | 0.96 | 0.67 | 646057 | SPRED2 | −0.90 | 0.81 |
| 743426 | KIAA1576 | 0.96 | 0.67 | 431301 | CHST2 | −0.90 | 0.81 |
| 307094 | GCAT | 0.96 | 0.67 | 1927991 | ENPP2 | −0.90 | 0.81 |
| 666371 | THBS1 | 0.95 | 0.67 | 1895676 | BARX1 | −0.90 | 0.81 |
| 81331 | FABP5 | 0.94 | 0.67 | 951303 | AA620527 | −0.90 | 0.81 |
| 282587 | CA11 | 0.94 | 0.67 | 1460653 | SEPT6 | −0.89 | 0.81 |
| 283995 | PAR1 | 0.94 | 0.67 | 810612 | S100A11 | −0.89 | 0.81 |
| 251019 | CDH1 | 0.94 | 0.67 | 60249 | SFTPC | −0.89 | 0.81 |
| 359684 | ZDHHC22 | 0.94 | 0.67 | 294537 | RAB17 | −0.89 | 0.81 |
| 502664 | RIS1 | 0.94 | 0.67 | 1324885 | LOC284542 | −0.89 | 0.81 |
| 681865 | C13orf25 | 0.93 | 0.67 | 756931 | S100A1 | −0.89 | 0.81 |
| 230882 | PAX6 | 0.93 | 0.67 | 1585518 | KIAA1442 | −0.88 | 0.81 |
| 768448 | JPH4 | 0.93 | 0.67 | 379598 | TRPV4 | −0.87 | 0.81 |
| 502446 | DNAPTP6 | 0.93 | 0.67 | 813631 | TM7SF3 | −0.87 | 0.81 |
| 1911780 | TCF7L2 | 0.92 | 0.67 | 1630411 | TDE1 | −0.87 | 0.81 |
| 24271 | TOX | 0.92 | 0.67 | 1456122 | THEA | −0.86 | 0.81 |
| 342640 | KIAA0101 | 0.92 | 0.67 | 1925681 | SMYD2 | −0.86 | 0.81 |
| 141758 | Hs.191591 | 0.92 | 0.67 | 133273 | PMP22 | −0.86 | 0.81 |
| 434768 | FST | 0.91 | 0.67 | 81316 | ARG99 | −0.86 | 0.81 |
| 782835 | FOXO1A | 0.91 | 0.67 | 81409 | GABARAPL1 | −0.86 | 0.81 |
| 147925 | Hs.298258 | 0.90 | 0.67 | 359835 | SAT | −0.85 | 0.81 |
| 878627 | AA775288 | 0.89 | 0.81 | 2010319 | NALP1 | −0.85 | 0.81 |
| 877789 | LYPDC1 | 0.88 | 0.81 | 1946438 | TM7SF3 | −0.85 | 0.81 |
| 137535 | TIF1 | 0.88 | 0.81 | 753467 | SLC2A3 | −0.85 | 0.81 |
| 282977 | ADCY2 | 0.88 | 0.81 | 435566 | NOS3AS | −0.85 | 0.81 |
| 1551722 | AA922660 | 0.88 | 0.81 | 42893 | R59724 | −0.84 | 0.81 |
| 743829 | RGMA | 0.88 | 0.81 | 154172 | FCGBP | −0.84 | 0.81 |
| 122982 | EGLN3 | 0.88 | 0.81 | 782145 | TPTE | −0.84 | 0.81 |
| 470092 | LARGE | 0.88 | 0.81 | 795841 | FLJ14466 | −0.84 | 0.81 |
| 192543 | KIAA0773 | 0.87 | 0.81 | 796398 | PEG3 | −0.84 | 0.81 |
| 1912578 | PTGIS | 0.87 | 0.81 | 754017 | C12orf4 | −0.83 | 0.81 |
| 810041 | SS18 | 0.86 | 0.81 | 340745 | Hs.371609 | −0.83 | 0.81 |
| 68265 | AFP | 0.86 | 0.81 | 898298 | PRKAB2 | −0.83 | 0.81 |
| 789369 | ID4 | 0.86 | 0.81 | 1558625 | Hs.371609 | −0.83 | 0.81 |
| 1534890 | ANKRD12 | 0.86 | 0.81 | 789253 | PSEN2 | −0.83 | 0.81 |
| 770462 | CPZ | 0.86 | 0.81 | 357298 | Hs.550621 | −0.83 | 1.12 |
| 758298 | TOX | 0.85 | 0.81 | 1554451 | GJC1 | −0.83 | 1.12 |
| 417800 | Hs.59203 | 0.85 | 0.81 | 795758 | DKFZP434B044 | −0.82 | 1.12 |
| 797059 | AA463250 | 0.85 | 0.81 | 825343 | MGC15887 | −0.82 | 1.12 |
| 341328 | TPM1 | 0.84 | 0.81 | 897865 | MID1 | −0.82 | 1.12 |
| 34934 | R45160 | 0.84 | 0.81 | 683569 | AA215397 | −0.82 | 1.12 |
| 812277 | PLXDC2 | 0.84 | 0.81 | 252663 | CALB1 | −0.82 | 1.12 |
| 281908 | COL8A1 | 0.84 | 0.81 | 306933 | C9orf25 | −0.82 | 1.12 |
| 504337 | HESX1 | 0.83 | 0.81 | 461690 | ACTR1B | −0.82 | 1.12 |
| 796569 | C17 | 0.83 | 0.81 | 2009885 | BCAT1 | −0.81 | 1.12 |
| 825369 | VGLL4 | 0.83 | 0.81 | 486493 | GPR124 | −0.81 | 1.12 |
| 809707 | JUNB | 0.83 | 0.81 | 510576 | AGR2 | −0.81 | 1.12 |
| 2306765 | C18orf43 | 0.83 | 0.81 | 841655 | JARID1A | −0.81 | 1.12 |
| 40963 | Hs.171485 | 0.83 | 0.81 | 564803 | FOXM1 | −0.81 | 1.12 |
| 151477 | FLJ38507 | 0.82 | 0.81 | 324785 | P4HA2 | −0.81 | 1.12 |
| 2010012 | LRRC17 | 0.82 | 0.81 | 826103 | AA521416 | −0.81 | 1.12 |
| 132637 | GCA | 0.82 | 0.81 | 66978 | T67547 | −0.81 | 1.12 |
| 309864 | JUNB | 0.82 | 0.81 | 1632011 | NPR2 | −0.80 | 1.12 |
| 753162 | TBC1D4 | 0.82 | 0.81 | 854189 | AA669383 | −0.80 | 1.12 |
| 51255 | Hs.126110 | 0.82 | 0.81 | 279496 | DND1 | −0.80 | 1.12 |
| 32962 | Hs.22545 | 0.81 | 0.81 | 45623 | SMYD2 | −0.80 | 1.12 |
| 782688 | DNALI1 | 0.81 | 0.81 | 1322814 | AA745659 | −0.80 | 1.12 |
| 436070 | CA14 | 0.81 | 0.81 | 744001 | RBM5 | −0.80 | 1.12 |
| 202535 | H19 | 0.80 | 1.12 | 305895 | Hs.180171 | −0.79 | 1.12 |
| 811028 | VMP1 | 0.80 | 1.12 | 491232 | PSEN2 | −0.79 | 1.12 |
| 144834 | MAP7 | 0.80 | 1.12 | 1492891 | ARF4L | −0.79 | 1.12 |
| 814769 | MLF1IP | 0.80 | 1.12 | 51548 | H20826 | −0.79 | 1.12 |
| 447786 | AUTS2 | 0.80 | 1.12 | 1588349 | IMPA3 | −0.79 | 1.12 |
| 727268 | Hs.545676 | 0.80 | 1.12 | 121981 | SLC2A14 | −0.79 | 1.12 |
| 971188 | AA774927 | 0.80 | 1.12 | 878572 | NET-5 | −0.79 | 1.12 |
| 810218 | OCIAD2 | 0.80 | 1.12 | 2018581 | IL6ST | −0.79 | 1.12 |
| 50114 | PCDHA6 | 0.80 | 1.12 | 154138 | MBTPS2 | −0.79 | 1.34 |
| 878630 | NBEA | 0.79 | 1.12 | 853962 | AA644695 | −0.79 | 1.34 |
| 360787 | TIF1 | 0.79 | 1.12 | 1916973 | NDUFA9 | −0.79 | 1.34 |
| 52430 | SALL2 | 0.79 | 1.12 | 49145 | Hs.494030 | −0.79 | 1.34 |
| 1696831 | AI095794 | 0.79 | 1.12 | 1554439 | Hs.550811 | −0.79 | 1.34 |
| 760231 | USP9X | 0.79 | 1.12 | 1475308 | Hs.546579 | −0.78 | 1.34 |
| 221295 | ID2 | 0.79 | 1.12 | 131979 | EPAS1 | −0.78 | 1.34 |
| 345601 | D2S448 | 0.79 | 1.12 | 1455745 | ZDHHC9 | −0.78 | 1.34 |
| 897656 | FARP1 | 0.79 | 1.12 | 768944 | PGK1 | −0.78 | 1.34 |
| 813265 | NFIB | 0.79 | 1.12 | 757152 | ZNF318 | −0.78 | 1.34 |
| 27069 | SCLY | 0.78 | 1.12 | 162199 | PTPRM | −0.78 | 1.34 |
| 809694 | CRABP1 | 0.78 | 1.12 | 855786 | WARS | −0.78 | 1.34 |
| 726779 | CNN1 | 0.78 | 1.34 | 502778 | LRP6 | −0.78 | 1.34 |
| 279577 | Hs.46551 | 0.77 | 1.34 | 1434905 | HOXB7 | −0.78 | 1.34 |
| 280758 | TMSB4Y | 0.77 | 1.34 | 489677 | UPP1 | −0.77 | 1.34 |
| 35626 | SLC38A1 | 0.77 | 1.34 | 124071 | ASB9 | −0.77 | 1.34 |
| 252830 | H88050 | 0.77 | 1.34 | 296020 | Hs.522906 | −0.77 | 1.34 |
| 854879 | SPHK2 | 0.77 | 1.34 | 191516 | CREBBP | −0.77 | 1.34 |
| 882402 | KIAA0692 | 0.77 | 1.34 | 380620 | PSEN2 | −0.77 | 1.34 |
| 486436 | UGP2 | 0.77 | 1.34 | 1732666 | AI191823 | −0.77 | 1.34 |
| 31475 | SALL3 | 0.77 | 1.34 | 825270 | PREX1 | −0.77 | 1.34 |
| 666451 | PSD3 | 0.77 | 1.34 | 247546 | VTN | −0.77 | 1.34 |
| 379709 | LRRN1 | 0.76 | 1.34 | 77651 | HDAC6 | −0.77 | 1.34 |
| 628357 | ACTN3 | 0.76 | 1.34 | 1637233 | TFCP2L1 | −0.77 | 1.34 |
| 2314305 | CDKN1C | 0.76 | 1.34 | 1323328 | PTHR1 | −0.77 | 1.34 |
| 1567985 | AA975922 | 0.76 | 1.34 | 586803 | PGF | −0.76 | 1.34 |
| 344036 | BNC2 | 0.76 | 1.34 | 377560 | CD3D | −0.76 | 1.34 |
| 843036 | MAP7 | 0.76 | 1.34 | 1470131 | TFCP2L1 | −0.76 | 1.34 |
| 782737 | USP44 | 0.76 | 1.34 | 83444 | SLC10A1 | −0.76 | 1.34 |
| 341310 | FRZB | 0.76 | 2.27 | 154600 | PLCD1 | −0.76 | 1.34 |
| 731025 | PPM1E | 0.75 | 2.27 | 1472405 | S100A10 | −0.76 | 1.34 |
| 282717 | BCL2 | 0.75 | 2.27 | 1456120 | GRK5 | −0.76 | 1.34 |
| 50354 | OTX2 | 0.74 | 2.27 | 214996 | FRS2 | −0.76 | 2.27 |
| 755444 | TMSB4X | 0.74 | 2.27 | 85313 | CCPG1 | −0.75 | 2.27 |
| 289936 | Hs.390594 | 0.74 | 2.27 | 295831 | DERA | −0.75 | 2.27 |
| 27396 | GAL3ST3 | 0.74 | 2.27 | 296623 | Hs.431518 | −0.75 | 2.27 |
| 788667 | PLEKHA9 | 0.74 | 2.27 | 711918 | QPCT | −0.75 | 2.27 |
| 1049291 | OR7E47P | 0.74 | 2.27 | 1732811 | TULP3 | −0.75 | 2.27 |
| 328542 | GALNT3 | 0.74 | 2.27 | 784296 | NR3C2 | −0.75 | 2.27 |
| 725395 | UBE2L6 | 0.73 | 2.27 | 809719 | URB | −0.75 | 2.27 |
| 1895357 | AI299356 | 0.73 | 2.27 | 284076 | CREBL2 | −0.75 | 2.27 |
| 1456776 | CLDN4 | 0.73 | 2.27 | 1552602 | PHKA1 | −0.74 | 2.27 |
| 758088 | CALD1 | 0.73 | 2.27 | 756595 | S100A10 | −0.74 | 2.27 |
| 340657 | LEFTY2 | 0.73 | 2.27 | 682418 | ELF4 | −0.74 | 2.27 |
| 365147 | ERBB2 | 0.73 | 2.27 | 811072 | Hs.217583 | −0.74 | 2.27 |
| 1855229 | Hs.149796 | 0.73 | 2.27 | 488301 | LOC149603 | −0.74 | 2.27 |
| 753291 | C1orf21 | 0.73 | 2.27 | 752557 | GPSM3 | −0.74 | 2.27 |
| 50499 | MGC72075 | 0.73 | 2.27 | 567127 | FLJ20716 | −0.74 | 2.27 |
| 126458 | MT1K | 0.72 | 2.27 | 1555659 | AI147534 | −0.74 | 2.27 |
| 740851 | Hs.479288 | 0.72 | 2.27 | 897301 | CMAS | −0.74 | 2.27 |
| 609155 | LRRN1 | 0.72 | 2.27 | 754559 | C2orf27 | −0.73 | 2.27 |
| 324437 | CXCL1 | 0.72 | 2.70 | 23819 | ABCG1 | −0.73 | 2.27 |
| 203003 | NME4 | 0.72 | 2.70 | 1917493 | SCAND2 | −0.73 | 2.27 |
| 566597 | PRSS16 | 0.72 | 2.70 | 753775 | GMPR | −0.73 | 2.27 |
| 194706 | USP9X | 0.72 | 2.70 | 1558655 | ASRGL1 | −0.73 | 2.27 |
| 783729 | ERBB2 | 0.72 | 2.70 | 1858444 | MDM4 | −0.73 | 2.27 |
| 755689 | RARG | 0.72 | 2.70 | 454341 | MYL4 | −0.73 | 2.27 |
| 214858 | LDB2 | 0.72 | 2.70 | 813520 | BPHB3 | −0.73 | 2.27 |
| 149743 | C15orf29 | 0.72 | 2.70 | 293336 | N64734 | −0.73 | 2.27 |
| 137387 | TFAP2A | 0.71 | 2.70 | 289794 | C12orf2 | −0.73 | 2.27 |
| 626793 | NIPA2 | 0.71 | 2.70 | 1526826 | HOXB2 | −0.73 | 2.27 |
| 858401 | SCG3 | 0.71 | 2.70 | 1126568 | Hs.116314 | −0.73 | 2.27 |
| 80643 | EDIL3 | 0.71 | 2.70 | 397488 | TBX3 | −0.73 | 2.27 |
| 1551239 | FLJ10884 | 0.71 | 2.70 | 713566 | MSP | −0.72 | 2.27 |
| 39824 | UNC13A | 0.71 | 2.70 | 267460 | CGI-141 | −0.72 | 2.27 |
| 301878 | SCGB3A2 | 0.71 | 2.70 | 1570663 | FKBP4 | −0.72 | 2.70 |
| 1605321 | C20orf24 | 0.71 | 2.70 | 1585211 | Hs.194678 | −0.72 | 2.70 |
| 277165 | TMEFF1 | 0.71 | 2.70 | 259884 | GPR126 | −0.71 | 2.70 |
| 347520 | BOC | 0.71 | 2.70 | 148469 | TYROBP | −0.71 | 2.70 |
| 812088 | NLN | 0.71 | 2.70 | 1855351 | EPSTI1 | −0.71 | 2.70 |
| 1607198 | FSIP1 | 0.71 | 2.70 | 1476466 | KBTBD9 | −0.71 | 2.70 |
| 1500643 | SLC13A1 | 0.71 | 2.70 | 298189 | Hs.171806 | −0.71 | 2.70 |
| 298702 | APOM | 0.70 | 2.70 | 940994 | Hs.105316 | −0.71 | 2.70 |
| 347035 | KIAA0476 | 0.70 | 2.70 | 1588935 | PHLDA3 | −0.71 | 2.70 |
| 293569 | C1orf21 | 0.70 | 2.70 | 346696 | TEAD4 | −0.70 | 2.70 |
| 309447 | TM4SF10 | 0.70 | 2.70 | 304975 | KIAA0318 | −0.70 | 2.70 |
| 22778 | R38615 | 0.70 | 2.70 | 45464 | AK2 | −0.70 | 2.70 |
| 324690 | GREM1 | 0.70 | 2.70 | 143997 | PSMD10 | −0.70 | 2.70 |
| 134712 | SLC7A1 | 0.70 | 2.70 | 789147 | ENO2 | −0.70 | 2.70 |
| 785941 | ZNF278 | 0.70 | 2.70 | 949939 | PGK1 | −0.70 | 2.70 |
| 34901 | DOK5 | 0.70 | 2.70 | 210789 | AGT | −0.70 | 2.70 |
| 491311 | EGLN3 | 0.70 | 2.70 | 1865128 | PEX5 | −0.70 | 2.70 |
| 41103 | TTYH1 | 0.70 | 2.70 | 730150 | LOC144363 | −0.70 | 2.70 |
| 813608 | Hs.346566 | 0.70 | 2.70 | 727251 | CD9 | −0.70 | 2.70 |
| 257109 | USP9X | 0.69 | 2.70 | 281053 | C2orf18 | −0.70 | 2.70 |
| 488207 | T1A-2 | 0.69 | 2.70 | 743810 | CDCA3 | −0.70 | 2.70 |
| 782826 | BACH | 0.69 | 2.70 | 280970 | NOL1 | −0.69 | 2.99 |
| 417226 | MYC | 0.69 | 2.70 | 361456 | DDIT3 | −0.69 | 2.99 |
| 323238 | CXCL1 | 0.69 | 2.70 | 271219 | Hs.487393 | −0.69 | 2.99 |
| 37980 | ZIC2 | 0.69 | 2.70 | 1682167 | MGC5370 | −0.69 | 2.99 |
| 628955 | FOXO1A | 0.69 | 2.70 | 283089 | LOC340542 | −0.69 | 2.99 |
| 1472735 | MT1E | 0.69 | 2.70 | 1635359 | RASD1 | −0.68 | 2.99 |
| 813628 | SCN2B | 0.69 | 2.70 | 309776 | CFLAR | −0.68 | 2.99 |
| 45542 | IGFBP5 | 0.69 | 2.70 | 206795 | ASGR2 | −0.68 | 2.99 |
| 141768 | ERBB2 | 0.69 | 2.99 | 40871 | C3F | −0.68 | 2.99 |
| 701115 | C6orf115 | 0.69 | 2.99 | 742642 | MIG-6 | −0.68 | 2.99 |
| 1635970 | MFHAS1 | 0.69 | 2.99 | 202498 | IL10RB | −0.68 | 2.99 |
| 377461 | CAV1 | 0.69 | 2.99 | 855523 | GPX3 | −0.68 | 2.99 |
| 173228 | GMFB | 0.68 | 2.99 | 1587065 | RPESP | −0.68 | 2.99 |
| 739193 | CRABP1 | 0.68 | 2.99 | 767041 | FLJ41841 | −0.68 | 2.99 |
| 29828 | TGFB1I4 | 0.68 | 2.99 | 359982 | AA035669 | −0.68 | 2.99 |
| 842918 | FARP1 | 0.68 | 2.99 | 1692195 | KIFAP3 | −0.68 | 2.99 |
| 127486 | LDHD | 0.68 | 2.99 | 505243 | ITPR2 | −0.68 | 2.99 |
| 51920 | OSBPL1A | 0.68 | 2.99 | 949938 | CST3 | −0.68 | 2.99 |
| 51378 | Hs.31924 | 0.68 | 2.99 | 2010188 | CCL26 | −0.68 | 2.99 |
| 506060 | Hs.506182 | 0.67 | 2.99 | 1734754 | LEPREL2 | −0.68 | 2.99 |
| 1865374 | EFCBP2 | 0.67 | 2.99 | 142326 | FLJ90036 | −0.67 | 2.99 |
| 2052032 | MYO10 | 0.67 | 2.99 | 256947 | NRK | −0.67 | 2.99 |
| 752652 | TCF7L2 | 0.67 | 2.99 | 1562645 | NFKB2 | −0.67 | 2.99 |
| 1457205 | LOC152195 | 0.67 | 2.99 | 1168484 | KITLG | −0.67 | 2.99 |
| 50562 | C8orf4 | 0.67 | 2.99 | 1641822 | WBP11 | −0.67 | 2.99 |
| 133136 | DEK | 0.67 | 2.99 | 609929 | DDX47 | −0.67 | 2.99 |
| 844680 | TRD@ | 0.67 | 2.99 | 1476157 | PEX5 | −0.67 | 2.99 |
| 825382 | DCP2 | 0.67 | 2.99 | 433253 | FBP1 | −0.67 | 2.99 |
| 80823 | RPL10A | 0.67 | 2.99 | 1943018 | IRAK1 | −0.67 | 2.99 |
| 502287 | EMB | 0.67 | 2.99 | 134430 | C9orf13 | −0.67 | 2.99 |
| 809603 | PTMA | 0.67 | 2.99 | 143661 | NTN4 | −0.67 | 3.00 |
| 504461 | KMO | 0.67 | 2.99 | 853066 | AA668256 | −0.67 | 3.00 |
| 366848 | TCF7L2 | 0.67 | 2.99 | 753914 | ITPR2 | −0.66 | 3.00 |
| 207107 | CALD1 | 0.66 | 2.99 | 752808 | TMED4 | −0.66 | 3.00 |
| 74537 | AFP | 0.66 | 2.99 | 1586703 | GPR3 | −0.66 | 3.00 |
| 2020772 | TM7SF2 | 0.66 | 2.99 | 897987 | NDUFA9 | −0.66 | 3.00 |
| 970591 | HMGB1 | 0.66 | 2.99 | 429349 | RGS4 | −0.66 | 3.00 |
| 1475968 | TEAD2 | 0.66 | 2.99 | 813189 | TDE1 | −0.66 | 3.00 |
| 81408 | C13orf7 | 0.66 | 2.99 | 51373 | OMG | −0.66 | 3.00 |
| 244652 | SET | 0.66 | 2.99 | 194136 | H50971 | −0.66 | 3.00 |
| 1586535 | Hs.120204 | 0.66 | 2.99 | 429368 | TLX1 | −0.66 | 3.00 |
| 230100 | Hs.546672 | 0.66 | 2.99 | 859912 | TDE1 | −0.66 | 3.00 |
| 502155 | PTGIS | 0.66 | 2.99 | 1627688 | LMO6 | −0.66 | 3.00 |
| 293032 | TFAP2A | 0.66 | 2.99 | 80162 | RAD51C | −0.66 | 3.00 |
| 283398 | TM4SF10 | 0.66 | 2.99 | 877832 | AA625628 | −0.66 | 3.00 |
| 327593 | Hs.547695 | 0.66 | 2.99 | 1896981 | XCL1 | −0.66 | 3.00 |
| 208718 | ANXA1 | 0.66 | 3.00 | 1670954 | KIAA1363 | −0.65 | 3.00 |
| 265694 | OLFML2B | 0.66 | 3.00 | 1635221 | ETNK1 | −0.65 | 3.00 |
| 291448 | SILV | 0.65 | 3.00 | 1501914 | P4HB | −0.65 | 3.00 |
| 592594 | LRIG1 | 0.65 | 3.00 | 1879169 | RAB21 | −0.65 | 3.00 |
| 137984 | FLJ38507 | 0.65 | 3.00 | 813426 | TRIB2 | −0.65 | 3.00 |
| 1761751 | MAPK8IP1 | 0.65 | 3.00 | 727988 | CDW52 | −0.65 | 3.00 |
| 1881469 | Hs.547698 | 0.65 | 3.00 | 302632 | B7 | −0.65 | 3.00 |
| 134783 | COL11A1 | 0.65 | 3.00 | 869187 | EPAS1 | −0.65 | 3.00 |
| 726658 | NME3 | 0.65 | 3.00 | 52031 | LOC126731 | −0.65 | 3.00 |
| 239256 | FZD7 | 0.65 | 3.00 | 43865 | DNCI1 | −0.65 | 3.00 |
| 284007 | LOC152485 | 0.65 | 3.00 | 1724716 | TTLL3 | −0.65 | 3.00 |
| 788641 | AP1S2 | 0.64 | 3.00 | 124737 | CHST12 | −0.65 | 3.00 |
| 878583 | CABP1 | 0.64 | 3.00 | 234348 | MXD3 | −0.64 | 3.00 |
| 854570 | TEAD2 | 0.64 | 3.00 | 1500631 | DDIT3 | −0.64 | 3.00 |
| 714106 | PLAU | 0.64 | 3.00 | 1609537 | WNK1 | −0.64 | 3.00 |
| 880747 | MGC3036 | 0.64 | 3.00 | 328821 | CFC1 | −0.64 | 3.00 |
| 782576 | Hs.459026 | 0.64 | 3.00 | 842826 | RBBP4 | −0.64 | 3.00 |
| 47359 | EDN1 | 0.64 | 3.00 | 2308429 | PPFIA4 | −0.64 | 3.00 |
| 1475734 | TOX | 0.64 | 3.00 | 1566554 | PRKAB2 | −0.64 | 3.00 |
| 1857589 | AI269390 | 0.64 | 3.00 | 810552 | REA | −0.64 | 3.00 |
| 1604674 | ZIC2 | 0.64 | 3.00 | 253733 | FOXC1 | −0.64 | 3.00 |
| 1574074 | KIAA1586 | 0.64 | 3.00 | 357190 | MGC8902 | −0.64 | 3.00 |
| 453602 | CALD1 | 0.64 | 3.00 | 162310 | PMP22 | −0.64 | 3.00 |
| 814353 | AA458838 | 0.64 | 3.00 | 1695674 | HSPB6 | −0.64 | 3.00 |
| 1700916 | C9orf39 | 0.64 | 3.00 | 289570 | NSMAF | −0.64 | 3.00 |
| 1948377 | OPRS1 | 0.64 | 3.00 | 66327 | CR1L | −0.64 | 3.00 |
| 740925 | INDO | 0.64 | 3.00 | 345103 | EPHB2 | −0.64 | 3.00 |
| 179266 | CTXN1 | 0.64 | 3.00 | 687667 | Hs.537002 | −0.64 | 3.66 |
| 79935 | T61475 | 0.64 | 3.00 | 856447 | IFI30 | −0.64 | 3.66 |
| 24415 | TP53 | 0.64 | 3.00 | 297212 | ITLN1 | −0.64 | 3.66 |
| 1897950 | C15orf29 | 0.64 | 3.00 | 1558505 | LEPRE1 | −0.64 | 3.66 |
| 627226 | SLC30A1 | 0.63 | 3.00 | 1473168 | ZC3HDC6 | −0.64 | 3.66 |
| 1492411 | EIF5A | 0.63 | 3.00 | 1661677 | RIF1 | −0.63 | 3.66 |
| 854581 | TCF4 | 0.63 | 3.00 | 1636900 | AI000268 | −0.63 | 3.66 |
| 241985 | PAR1 | 0.63 | 3.00 | 345916 | SPTBN1 | −0.63 | 3.66 |
| 1606557 | FHL2 | 0.63 | 3.00 | 395400 | MBD6 | −0.63 | 3.66 |
| 276574 | FLJ36754 | 0.63 | 3.66 | 279970 | ADORA2A | −0.63 | 3.66 |
| 366093 | ZNF397 | 0.63 | 3.66 | 1671108 | AI075256 | −0.63 | 3.66 |
| 1605008 | IGSF4C | 0.63 | 3.66 | 133988 | ACSL4 | −0.63 | 3.66 |
| 1160531 | ERBB3 | 0.63 | 3.66 | 377987 | ADAMTS15 | −0.63 | 3.66 |
| 565075 | STC1 | 0.63 | 3.66 | 729964 | SMPD1 | −0.63 | 3.66 |
| 1570558 | AA932334 | 0.63 | 3.66 | 2009974 | ACHE | −0.63 | 3.66 |
| 739155 | CDH6 | 0.63 | 3.66 | 812961 | SIPA1L2 | −0.63 | 3.66 |
| 739159 | BPHL | 0.63 | 3.66 | 810743 | MLF2 | −0.63 | 3.66 |
| 488246 | KIAA1913 | 0.63 | 3.66 | 1554420 | TCEA2 | −0.63 | 3.66 |
| 137297 | PGAP1 | 0.63 | 3.66 | 132702 | P4HB | −0.63 | 3.66 |
| 271670 | TNFSF13 | 0.63 | 3.66 | 1589083 | DEFB1 | −0.62 | 3.66 |
| 324307 | TM4SF10 | 0.63 | 3.66 | 1644045 | TULP3 | −0.62 | 3.66 |
| 347331 | SNTB1 | 0.63 | 3.66 | 770785 | MAN1C1 | −0.62 | 3.66 |
| 282895 | LRRC16 | 0.62 | 3.66 | 1475648 | TTN | −0.62 | 3.66 |
| 250678 | FLJ20171 | 0.62 | 3.66 | 299603 | AI822111 | −0.62 | 3.66 |
| 1371759 | CUGBP2 | 0.62 | 3.66 | 1917063 | SDSL | −0.62 | 3.66 |
| 725365 | GAS1 | 0.62 | 3.66 | 1759254 | STS-1 | −0.62 | 3.66 |
| 2005924 | MATK | 0.62 | 3.66 | 127370 | R08549 | −0.62 | 3.66 |
| 795746 | MLF1IP | 0.62 | 3.66 | 26482 | ZNF335 | −0.62 | 3.66 |
| 1895737 | Hs.445295 | 0.62 | 3.66 | 811162 | FMOD | −0.62 | 3.66 |
| 742776 | YPEL1 | 0.62 | 3.66 | 79562 | MOSPD1 | −0.62 | 3.66 |
| 236338 | TP53 | 0.62 | 3.66 | 50166 | OATL1 | −0.62 | 3.66 |
| 686667 | GCDH | 0.62 | 3.66 | 1160995 | ERF | −0.62 | 3.66 |
| 180520 | UBE3A | 0.62 | 3.66 | 40040 | KIAA1126 | −0.61 | 3.66 |
| 447509 | HLA-DOA | 0.62 | 3.66 | 2296063 | KIAA0528 | −0.61 | 3.66 |
| 1862529 | Hs.433460 | 0.62 | 3.66 | | | | |
| 47460 | B3GAT1 | 0.62 | 3.66 | | | | |
| 345645 | PDGFB | 0.62 | 3.66 | | | | |
| 489169 | C10orf83 | 0.62 | 3.66 | | | | |
| 755299 | IER2 | 0.61 | 3.66 | | | | |
| 504774 | GGTLA1 | 0.61 | 3.66 | | | | |
| 1602927 | MGC35048 | 0.61 | 3.66 | | | | |
| 213850 | FJX1 | 0.61 | 3.66 | | | | |
| 38618 | Hs.530150 | 0.61 | 3.66 | | | | |
| 125187 | ERCC2 | 0.61 | 3.66 | | | | |
| 300099 | TM4SF9 | 0.61 | 3.66 | | | | |
| 153646 | R48843 | 0.61 | 3.66 | | | | |
| 768417 | EPB41L3 | 0.61 | 3.66 | | | | |
| 133518 | MAPRE2 | 0.61 | 3.66 | | | | |
| 1556401 | AA936454 | 0.61 | 3.66 | | | | |

By a simple ranking test (one-class significant analysis of microarrays), 328 genes were identified with highest level and 313 genes with lowest level expression in the ES cells.
Genes were selected according to the cut-off q value ≦0.05.

TABLE 2
Prostate cancer clinical data and ES type

| Clinical data, Lapointe et al., 2004 (Ref. # 62) | | | | | | | | | | |
|---|---|---|---|---|---|---|---|---|---|---|
| | | | | | | Recurrence- | | | | |
| | | | | | | free; | | This invention | | |
| Patient | | Gleason | | | | survival | | ES type (b) | | |
| ID (a) | Age | grade | Stage T | Node N | Metastasis M | (months) | Recurrence* | q ≦ 0.01 | q ≦ 0.05 | q ≦ 0.1 |
| PC229 | 47 | 3 + 3 | T2b | N0 | M0 | 0.03 | 0 | 1 | 1 | 1 |
| PC112 | 57 | 3 + 3 | T2b | N0 | M0 | 12.06 | 0 | 1 | 1 | 1 |
| PC083 | 63 | 4 + 4 | T3a | N0 | M0 | 13.6 | 0 | 1 | 1 | 1 |
| PC041 | 54 | 3 + 3 | T2b | N0 | M0 | 14.2 | 0 | 1 | 1 | 1 |
| PC191 | 59 | 3 + 3 | T3a | N0 | M0 | 15.5 | 0 | 1 | 1 | 1 |
| PC111 | 56 | 3 + 3 | T2b | N0 | M0 | 17.4 | 0 | 1 | 1 | 1 |
| PC187 | 58 | 3 + 3 | T2b | N0 | M0 | 2.5 | 0 | 1 | 1 | 1 |
| PC028 | 62 | 3 + 4 | T2b | N0 | M0 | 22.9 | 0 | 1 | 1 | 1 |
| PC335 | 58 | 3 + 4 | T3a | N0 | M0 | 5.6 | 0 | 1 | 1 | 1 |
| PC224 | 64 | 4 + 3 | T3a | N0 | M0 | 5.6 | 0 | 1 | 1 | 1 |
| PC100 | 67 | 4 + 4 | T2b | N0 | M0 | 9 | 0 | 0 | 1 | 1 |
| PC087 | 68 | 4 + 5 | T3a | N0 | M0 | 9.4 | 0 | 0 | 1 | 1 |
| PC087 | 60 | 4 + 4 | T3b | N0 | M0 | 16.2 | 1 | 1 | 1 | 1 |
| PC168 | 50 | 4 + 5 | T2b | N0 | M0 | 17.1 | 1 | 1 | 1 | 1 |
| PC019 | 57 | 4 + 5 | T3a | N1 | M0 | 19.1 | 1 | 1 | 1 | 1 |
| PC265 | 59 | 4 + 4 | T2b | N0 | M0 | 2.76 | 1 | 0 | 1 | 1 |
| PC007 | 56 | 3 + 3 | T2b | N0 | M0 | 27.7 | 1 | 1 | 1 | 1 |
| PC250 | 55 | 3 + 3 | T3b | N1 | M0 | 3.1 | 1 | 1 | 1 | 1 |
| PC103 | 61 | 4 + 3 | T3a | N0 | M0 | 5.9 | 1 | 1 | 1 | 1 |
| PC055 | 64 | 4 + 3 | T3b | N0 | M0 | N/A | N/A | 1 | 1 | 1 |
| PC130 | 58 | 3 + 4 | T3a | N0 | M0 | N/A | N/A | 1 | 1 | 1 |
| PC176 | 67 | 4 + 4 | T3b | N0 | M0 | N/A | N/A | 1 | 1 | 1 |
| PC235 | N/A | 3 + 3 | N/A | N/A | N/A | N/A | N/A | 1 | 1 | 1 |
| PC317 | 58 | 3 + 3 | T2 | N0 | Mx | N/A | N/A | 1 | 1 | 1 |
| PC014 | N/A | 3 + 3 | N/A | N/A | N/A | N/A | N/A | 1 | 1 | 1 |
| PC027 | 60 | LN meta | T3a | N1 | M0 | N/A | N/A | 1 | 1 | 1 |
| PC054 | 62 | 4 + 5 | T3b | N1 | M0 | N/A | N/A | 1 | 1 | 1 |
| PC057 | 61 | 3 + 4 | T2b | N0 | M0 | N/A | N/A | 1 | 1 | 1 |
| PC058 | 66 | 3 + 4 | T3b | N0 | M0 | N/A | N/A | 1 | 1 | 1 |
| PC114 | 62 | LN meta | T4 | Nx | Mx | N/A | N/A | 1 | 1 | 1 |
| PC115 | N/A | LN meta | N/A | N/A | N/A | N/A | N/A | 1 | 1 | 1 |
| PC116 | 58 | LN meta | T3 | N1 | M0 | N/A | N/A | 1 | 1 | 1 |
| PC118 | N/A | LN meta | N/A | N/A | N/A | N/A | N/A | 1 | 1 | 1 |
| PC122 | 66 | LN meta | T3 | N1 | M0 | N/A | N/A | 1 | 1 | 1 |
| PC129 | 63 | LN meta | T3 | N1 | M0 | N/A | N/A | 1 | 1 | 1 |
| PC133 | 55 | LN meta | T3 | N1 | M0 | N/A | N/A | 1 | 1 | 1 |
| PC171 | 50 | 3 + 3 | T3a | N0 | M0 | N/A | N/A | 1 | 1 | 1 |
| PC174 | 62 | 3 + 4 | T3b | N0 | M0 | N/A | N/A | 1 | 1 | 1 |
| PC180 | N/A | 3 + 4 | N/A | N/A | N/A | N/A | N/A | 1 | 1 | 1 |
| PC181 | 56 | 4 + 3 | T3a | N0 | M0 | N/A | N/A | 1 | 1 | 1 |
| PC194 | N/A | LN meta | N/A | N/A | N/A | N/A | N/A | 1 | 1 | 1 |
| PC308 | 59 | 4 + 5 | T3a | N0 | Mx | N/A | N/A | 1 | 1 | 1 |
| PC309 | 62 | 4 + 4 | T3a | N0 | Mx | N/A | N/A | 1 | 1 | 1 |
| PC310 | 72 | 4 + 3 | T3a | N0 | Mx | N/A | N/A | 1 | 1 | 1 |
| PC311 | 48 | 3 + 3 | T3a | N0 | Mx | N/A | N/A | 1 | 1 | 1 |
| PC312 | 59 | 3 + 3 | T2 | N0 | Mx | N/A | N/A | 1 | 1 | 1 |
| PC314 | 45 | 3 + 3 | T2 | N0 | Mx | N/A | N/A | 1 | 1 | 1 |
| PC315 | 65 | 4 + 4 | T3a | N0 | Mx | N/A | N/A | 1 | 1 | 1 |
| PC316 | 52 | 3 + 4 | T3a | N0 | Mx | N/A | N/A | 1 | 1 | 1 |
| PC319 | 58 | 4 + 4 | T3a | N1 | Mx | N/A | N/A | 1 | 1 | 1 |
| PC126 | 63 | 3 + 4 | T2a | N0 | M0 | N/A | N/A | 0 | 1 | 1 |
| PC138 | 60 | 4 + 4 | T3a | N0 | M0 | N/A | N/A | 0 | 1 | 1 |
| PC148 | 58 | 3 + 4 | T2b | N0 | M0 | 0.03 | 0 | 1 | 0 | 1 |
| PC205 | 66 | 3 + 4 | T2b | N0 | M0 | 0.03 | 0 | 1 | 0 | 1 |
| PC032 | N/A | 3 + 3 | T3b | N0 | M0 | 11.5 | 0 | 0 | 0 | 0 |
| PC215 | 62 | 3 + 3 | T2b | N0 | M0 | 12.3 | 0 | 0 | 0 | 0 |
| PC092 | 68 | 3 + 3 | T2b | N0 | M0 | 13.7 | 0 | 0 | 0 | 0 |
| PC102 | 48 | 3 + 3 | T2b | N1 | M0 | 16 | 0 | 1 | 0 | 1 |
| PC037 | 50 | 4 + 3 | T2b | N0 | M0 | 16.2 | 0 | 0 | 0 | 0 |
| PC195 | 55 | 3 + 4 | T2b | N0 | M0 | 5.8 | 0 | 0 | 0 | 0 |
| PC190 | 72 | 3 + 3 | T2b | N0 | M0 | 6.5 | 0 | 0 | 0 | 0 |
| PC021 | 61 | 3 + 3 | T2b | N0 | M0 | 9.8 | 0 | 0 | 0 | 0 |
| PC005 | N/A | 3 + 3 | N/A | N/A | N/A | N/A | N/A | 1 | 0 | 0 |
| PC177 | 57 | 3 + 4 | T2a | N0 | M0 | N/A | N/A | 0 | 0 | 0 |
| PC233 | N/A | 3 + 3 | N/A | N/A | N/A | N/A | N/A | 0 | 0 | 0 |
| PC313 | 50 | 3 + 4 | T2 | N0 | Mx | N/A | N/A | 0 | 0 | 0 |
| PC056 | 68 | 3 + 4 | T2b | N0 | M0 | N/A | N/A | 0 | 0 | 0 |
| PC173 | 72 | 3 + 3 | T3b | N0 | M0 | N/A | N/A | 0 | 0 | 0 |
| PC110 | 48 | 4 + 4 | T2b | N0 | M0 | N/A | N/A | 0 | 0 | 0 |
| PC153 | 64 | adenoid | T2b | N0 | M0 | N/A | N/A | 0 | 0 | 0 |
| | | cystic | | | | | | | | |
| PC318 | 56 | 4 + 3 | T3a | N0 | Mx | N/A | N/A | 0 | 0 | 0 |

LN meta: lymph node metastasis. N/A: non available.
(a) All patients hade one tumor sample analyzed. A fraction of patients hade also normal tissues from unaffected areas of the prostate analyzed; they are presented as the “normal” cluster in FIG. 2.
(b) Increasing the q value in the one-class SAM (significant analysis of microarrays) ranking test gave a list of increased number of significant ES genes as shown in FIG. 1. By choosing different q value cut-off at 0.01, 0.05 and 0.1, there were 201, 641 and 1386 significant ES genes selected respectively. Using the expression profile of these three gene lists to predict the tumor aggressiveness gave some slight different results as shown in this table. The result by the gene list at q ≦ 0.05 gave the best prediction.

TABLE 3
Lung adenocarcinoma clinical data and ES type

| Clinical and pathological data, Garber et al., 2001 | | | | | | | |
|---|---|---|---|---|---|---|---|
| (Ref. # 63) | | | | | This invention | | |
| | | | Survival | | ES type (b) | | |
| Patient (a) | Grade | Stage | (months) | Status | q ≦ 0.01 | q ≦ 0.05 | q ≦ 0.1 |
| 313-99 | 3 | pT2pN1pM1 | 17 | 1 | 0 | 0 | 0 |
| 198-96 | 2 | pT1pN2 | 1 | 1 | 0 | 0 | 0 |
| 199-97 | 2 | pT2pN1pM1 | 16 | 1 | 0 | 0 | 0 |
| 218-97 | 3 | pT2pN2 | 12 | 1 | 0 | 0 | 1 |
| 181-96 | 2 | pT4pN0 M1 | 25 | 1 | 0 | 0 | 1 |
| 204-97 | 2 | pT2pN2 M1 | 36 | 1 | 1 | 0 | 1 |
| 165-96 | 2 | pT1pN2 M1 | 18+ | 0 | 0 | 0 | 0 |
| 222-97 | 3 | pT2pN2 | 48+ | 0 | 0 | 0 | 0 |
| 226-97 | 3 | pT3pN2 | 48+ | 0 | 0 | 0 | 0 |
| 137-96 | 2 | pT2pN0 | 32 | 0 | 0 | 0 | 0 |
| 156-96 | 1 | pT2pN0 | 54+ | 0 | 0 | 0 | 0 |
| 180-96 | 2 | pT1pN0 | 54+ | 0 | 0 | 0 | 0 |
| 187-96 | 2 | pT1pN0 | 54+ | 0 | 0 | 0 | 0 |
| 185-96 | 2 | pT1pN0 M0 | 54+ | 0 | 0 | 0 | 0 |
| 132-95 | 3 | pT1pN0 | 37 | 0 | 0 | 0 | 1 |
| 320-00 | 3 | pT2pN1pM1 | | | 0 | 0 | 0 |
| 68-96 | 2 | pT1pN0 | | | 0 | 0 | 0 |
| 319-00PT | 2 | pT1pN2pM1 | | | 0 | 0 | 1 |
| Nov-00 | 2 | pT2pN0 | | | 1 | 0 | 1 |
| Dec-00 | 2 | pT1pN1 | | | 0 | 0 | 1 |
| 223-97 | 3 | pT2pN2 | 5 | 1 | 1 | 1 | 0 |
| 257-97 | 3 | pT2pN2 | 2 | 1 | 0 | 1 | 1 |
| 59-96 | 3 | pT2pN0 M1 | 11 | 1 | 1 | 1 | 1 |
| 80-96 | 3 | pT2pN2 M1 | 3 | 1 | 1 | 1 | 1 |
| 139-96 | 3 | pT3pN1pM1 | 5 | 1 | 1 | 1 | 1 |
| 184-96 | 2 | pT2pN2 M1 | 3 | 1 | 1 | 1 | 1 |
| 234-97 | 3 | pT2pN2pM1 | 0 | 1 | 1 | 1 | 1 |
| 265-98 | 2 | pT1 | 15 | 1 | 1 | 1 | 1 |
| 306-99 | 3 | pT2pN1 | 24+ | 0 | 1 | 1 | 1 |
| 319-00MT | 3 | | | | 0 | 1 | 0 |
| 178-96 | 2 | pT2pN0 | | | 1 | 1 | 1 |

(a) Table 3 presents clinical data from lung adenocarcinoma cases only. In FIG. 3 cases with non-adenocarcinoma are included, comprising large cell lung cancer, small cell lung cancer, and squamous cell lung cancer. The non-adenocarcinoma cases were analyzed by gene expression profiling in the original publication but lacked clinical follow-up data.
(b) By choosing different q value cut-off at 0.01, 0.05 and 0.1, 201, 641, and 1386, respectively, significant ES genes were selected. Using the expression profile of the corresponding gene lists for tumor aggressiveness prediction provided slightly different results as shown Table 3. The q ≦ 0.05 gene list gave the best prediction.

TABLE 4
Gastric cancer clinical data and ES type

| Clinical and pathological data, Chen et al., 2003 (Ref. # 64) | | | | | | | | | This |
|---|---|---|---|---|---|---|---|---|---|
| Sample | | Tumor | | Tumor | | EBV | Survival | Survival, | invention |
| ID (a) | SEX | site | Tumor type | stage | H. pylori | ISH | status | months | ES type (b) |
| HKG11T | F | Antrum | Diffused | IVA | − | − | 1 | 2 | 1 |
| HKG38T | F | Cardia | Intestinal | IVA | − | − | 1 | 3 | 1 |
| HKG23T | M | Antrum | Intestinal | IVB | − | − | 1 | 3 | 1 |
| HKG68T | M | Cardia | Intestinal | IVB | + | − | 1 | 3 | 1 |
| HKG1T | F | Antrum | Diffused | IIIA | − | − | 1 | 4 | 1 |
| HKG55T | M | Antrum | Diffused | IIIB | − | − | 1 | 4 | 1 |
| HKG69T | F | Cardia | Intestinal | IIIB | − | − | 1 | 4 | 1 |
| HKG49T | F | Cardia | Mixed | IVA | + | − | 1 | 4 | 1 |
| HKG27T | F | Cardia | Intestinal | IIIB | − | − | 1 | 5 | 1 |
| HKG64T | M | Antrum | Intestinal | IIIA | + | − | 1 | 6 | 1 |
| HKG32T | F | Antrum | Intestinal | II | − | − | 1 | 8 | 1 |
| HKG53T | M | Cardia | Mixed | IVA | + | − | 1 | 8 | 1 |
| HKG2T | M | Antrum | Intestinal | IIIB | + | − | 1 | 10 | 1 |
| HKG31T | M | Cardia | Intestinal | IVA | − | − | 1 | 10 | 1 |
| HKG78T | M | Cardia | Mixed | IIIB | + | − | 1 | 10 | 1 |
| HKG42T | M | Body | Intestinal | IIIA | − | + | 1 | 12 | 1 |
| HKG30T | F | Body | Intestinal | IIIB | − | − | 1 | 12 | 1 |
| HKG44T | F | Antrum | Diffused | IIIA | + | − | 1 | 14 | 1 |
| HKG36T | M | Body | Intestinal | IIIA | + | − | 1 | 15 | 1 |
| HKG19T | M | Cardia | Intestinal | IVA | + | − | 1 | 20 | 1 |
| HKG34T | M | Cardia | Intestinal | IVA | + | − | 1 | 20 | 1 |
| HKG51T | F | Body | Mixed | IIIA | + | − | 1 | 21 | 1 |
| HKG6T | M | Antrum | Diffused | IIIA | + | − | 1 | 26 | 1 |
| HKG52T | F | Antrum | Diffused | IIIB | + | − | 1 | 27 | 1 |
| HKG9T | M | Cardia | Intestinal | IIIB | − | − | 1 | 27 | 1 |
| HKG8T | M | Body | Intestinal | IIIA | + | − | 1 | 29 | 1 |
| HKG35T | F | Antrum | Diffused | IIIA | − | − | 1 | 30 | 1 |
| HKG73T | M | Body | Intestinal | II | + | + | 1 | 32 | 1 |
| HKG61T | M | Body | Intestinal | IIIA | + | − | 1 | 38 | 1 |
| HKG87T | F | Antrum | Diffused | IIIA | − | − | 1 | 45 | 1 |
| HKG20T | M | Antrum | Diffused | IIIB | + | − | 1 | 45 | 1 |
| HKG18T | F | Antrum | Intestinal | II | + | − | 0 | 1 | 1 |
| HKG84T | F | Antrum | Intestinal | IIIA | + | − | 0 | 1 | 1 |
| HKG26T | M | Cardia | Intestinal | IIIB | + | + | 0 | 1 | 1 |
| HKG92T | M | Cardia | Intestinal | IB | − | − | 0 | 11 | 1 |
| HKG71T | M | Antrum | Diffused | IIIB | − | − | 0 | 16 | 1 |
| HKG90T | M | Antrum | Intestinal | IB | + | − | 0 | 18 | 1 |
| HKG76T | M | Cardia | Intestinal | IB | − | − | 0 | 27 | 1 |
| HKG74T | F | Body | Intestinal | IB | + | − | 0 | 28 | 1 |
| HKG77T | F | Antrum | Intestinal | II | + | − | 0 | 29 | 1 |
| HKG43T | F | Cardia | Intestinal | II | − | − | 0 | 32 | 1 |
| HKG70T | M | Antrum | Intestinal | II | + | − | 0 | 34 | 1 |
| HKG67T | M | Antrum | Intestinal | IIIA | + | − | 0 | 37 | 1 |
| HKG66T | M | Antrum | Intestinal | II | − | − | 0 | 38 | 1 |
| HKG63T | F | Antrum | Intestinal | II | + | − | 0 | 42 | 1 |
| HKG3T | M | Antrum | Intestinal | IB | + | − | 0 | 45 | 1 |
| HKG58T | M | Antrum | Intestinal | II | + | − | 0 | 46 | 1 |
| HKG22T | F | Cardia | Mixed | IB | − | − | 0 | 51 | 1 |
| HKG33T | M | Antrum | Mixed | IIIA | − | − | 0 | 51 | 1 |
| HKG15T | F | Antrum | Mixed | IB | + | − | 0 | 57 | 1 |
| HKG13T | M | Antrum | Intestinal | II | − | − | 0 | 91 | 1 |
| HKG29T | F | Body | Intestinal | IIIA | − | − | N/A | N/A | 0b |
| HKG57T | M | Antrum | Diffused | IVA | − | − | 1 | 2 | 0a |
| HKG21T | M | Body | Intestinal | IIIA | + | + | 1 | 5 | 0b |
| HKG5T | M | Cardia | Intestinal | IIIB | − | − | 1 | 6 | 0a |
| HKG25T | M | Cardia | Intestinal | IVB | − | − | 1 | 8 | 0b |
| HKG60T | M | Body | Intestinal | IVA | + | + | 1 | 10 | 0b |
| HKG41T | F | Antrum | Intestinal | IIIA | − | − | 1 | 13 | 0a |
| HKG39T | F | Cardia | Intestinal | IIIA | + | − | 1 | 14 | 0a |
| HKG89T | M | Cardia | Intestinal | IVB | + | + | 1 | 15 | 0b |
| HKG16T | M | Antrum | Intestinal | IIIA | − | − | 1 | 16 | 0a |
| HKG82T | F | Antrum | Intestinal | IIIB | + | − | 1 | 17 | 0a |
| HKG48T | F | Cardia | Intestinal | IVA | − | − | 1 | 18 | 0a |
| HKG17T | F | Diffused | Diffused | IIIB | − | − | 1 | 20 | 0b |
| HKG24T | F | Antrum | indeterminate | IIIA | + | − | 1 | 20 | 0a |
| HKG37T | M | Cardia | Intestinal | IB | − | − | 1 | 43 | 0a |
| HKG79T | F | Antrum | Intestinal | IB | − | − | 0 | 1 | 0a |
| HKG45T | M | Body | Intestinal | IIIB | + | + | 0 | 1 | 0b |
| HKG47T | M | Cardia | Intestinal | IIIB | − | − | 0 | 2 | 0b |
| HKG10T | F | Body | Intestinal | IIIB | + | + | 0 | 3 | 0b |
| HKG94T | M | Body | Intestinal | II | − | + | 0 | 9 | 0b |
| HKG93T | F | Body | Intestinal | IB | + | + | 0 | 11 | 0b |
| HKG81T | F | Antrum | Intestinal | II | − | − | 0 | 12 | 0a |
| HKG91T | M | Cardia | Intestinal | IB | + | − | 0 | 18 | 0b |
| HKG75T | F | Cardia | Intestinal | II | − | − | 0 | 21 | 0a |
| HKG83T | F | Antrum | Intestinal | II | − | − | 0 | 21 | 0a |
| HKG28T | M | Cardia | Intestinal | IIIA | − | − | 0 | 22 | 0a |
| HKG72T | F | Antrum | Intestinal | II | + | − | 0 | 31 | 0a |
| HKG80T | M | Antrum | Intestinal | II | + | − | 0 | 32 | 0a |
| HKG65T | F | Antrum | Diffused | IIIA | + | − | 0 | 38 | 0b |
| HKG59T | M | Antrum | Intestinal | II | + | − | 0 | 41 | 0a |
| HKG40T | F | Body | Intestinal | II | − | − | 0 | 43 | 0a |
| HKG62T | F | Antrum | Intestinal | IVA | + | − | 0 | 44 | 0a |
| HKG54T | M | Cardia | Intestinal | IIIA | − | + | 0 | 49 | 0b |
| HKG56T | M | Body | Intestinal | IIIA | + | + | 0 | 49 | 0b |
| HKG7T | F | Body | Mixed | IIIA | + | − | 0 | 51 | 0b |
| HKG14T | M | Body | Intestinal | IB | + | − | 0 | 71 | 0a |
| HKG46T | M | Body | Intestinal | IA | + | − | 0 | 77 | 0b |
| HKG12T | M | Antrum | Intestinal | IIIB | − | − | 0 | 87 | 0a |

(a) Only tumor sample ID was indicated in Table 4. Some cases had both a tumor sample and a normal sample from respective stomach areas analyzed by gene expression profiling. The normal samples formed a normal cluster as shown in FIG. 5.
(b) The ES type was determined by using the gene list of 641 ES predictor genes selected at q ≦ 0.05 in the one-class SAM.

TABLE 5
Leukemia clinical data and ES type

| Clinical data, Bullinger et al., 2004 (Ref. # 66) | | | | This |
|---|---|---|---|---|
| | | | Overall | invention |
| Sample ID | Cytogenetic group | Status | survival (days) | ES type (a) |
| AML 26 | t(8; 21) | alive | 138 | 1 |
| AML 71 | other | alive | 138 | 1 |
| AML 49 | normal karyotype | alive | 211 | 1 |
| AML 105 | t(8; 21) | alive | 211 | 1 |
| AML 75 | normal karyotype | alive | 238 | 1 |
| AML 47 | del(7q)/-7 | alive | 281 | 1 |
| AML 94 | normal karyotype | alive | 359 | 1 |
| AML 44 | t(8; 21) | alive | 509 | 1 |
| AML 30 | normal karyotype | alive | 515 | 1 |
| AML 16 | t(8; 21) | alive | 610 | 1 |
| AML 114 | t(8; 21) | alive | 611 | 1 |
| AML 51 | del(7q)/-7 | alive | 622 | 1 |
| AML 48 | t(8; 21) | alive | 836 | 1 |
| AML 115 | normal karyotype | alive | 1107 | 1 |
| AML 107 | +8sole | dead | 7 | 1 |
| AML 58 | del(7q)/-7 | dead | 12 | 1 |
| AML 98 | t(8; 21) | dead | 15 | 1 |
| AML 78 | complex karyotype | dead | 21 | 1 |
| AML 42 | normal karyotype | dead | 31 | 1 |
| AML 57 | normal karyotype | dead | 32 | 1 |
| AML 52 | del(7q)/-7 | dead | 33 | 1 |
| AML 24 | complex karyotype | dead | 35 | 1 |
| AML 92 | del(7q)/-7 | dead | 44 | 1 |
| AML 56 | normal karyotype | dead | 75 | 1 |
| AML 13 | normal karyotype | dead | 85 | 1 |
| AML 118 | normal karyotype | dead | 99 | 1 |
| AML 102 | normal karyotype | dead | 102 | 1 |
| AML 62 | t(8; 21) | dead | 126 | 1 |
| AML 113 | normal karyotype | dead | 142 | 1 |
| AML 39 | normal karyotype | dead | 146 | 1 |
| AML 61 | normal karyotype | dead | 182 | 1 |
| AML 93 | normal karyotype | dead | 203 | 1 |
| AML 4 | t(8; 21) | dead | 210 | 1 |
| AML 5 | complex karyotype | dead | 243 | 1 |
| AML 76 | normal karyotype | dead | 250 | 1 |
| AML 96 | normal karyotype | dead | 273 | 1 |
| AML 45 | normal karyotype | dead | 291 | 1 |
| AML 87 | normal karyotype | dead | 316 | 1 |
| AML 18 | other | dead | 323 | 1 |
| AML 80 | del(7q)/-7 | dead | 333 | 1 |
| AML 67 | +8sole | dead | 414 | 1 |
| AML 66 | del(7q)/-7 | dead | 470 | 1 |
| AML 41 | other | dead | 540 | 1 |
| AML 17 | normal karyotype | dead | 570 | 1 |
| AML 46 | normal karyotype | dead | 663 | 1 |
| AML 108 | normal karyotype | dead | 672 | 1 |
| AML 14 | del(7q)/-7 | dead | 711 | 1 |
| AML 8 | normal karyotype | alive | 206 | 0a |
| AML 116 | normal karyotype | alive | 271 | 0a |
| AML 72 | complex karyotype | alive | 297 | 0a |
| AML 25 | inv(16) | alive | 400 | 0a |
| AML 34 | inv(16) | alive | 422 | 0a |
| AML 9 | normal karyotype | alive | 438 | 0a |
| AML 53 | inv(16) | alive | 493 | 0a |
| AML 84 | inv(16) | alive | 511 | 0a |
| AML 112 | normal karyotype | alive | 524 | 0a |
| AML 70 | inv(16) | alive | 551 | 0a |
| AML 89 | inv(16) | alive | 609 | 0a |
| AML 12 | normal karyotype | alive | 610 | 0a |
| AML 55 | normal karyotype | alive | 688 | 0a |
| AML 35 | normal karyotype | alive | 689 | 0a |
| AML 90 | inv(16) | alive | 690 | 0a |
| AML 109 | normal karyotype | alive | 720 | 0a |
| AML 81 | inv(16) | alive | 839 | 0a |
| AML 20 | t(9; 11) | alive | 884 | 0a |
| AML 65 | inv(16) | alive | 980 | 0a |
| AML 43 | normal karyotype | alive | 987 | 0a |
| AML 50 | t(9; 11) | alive | 1296 | 0a |
| AML 79 | inv(16) | alive | 1388 | 0a |
| AML 97 | inv(16) | alive | 1625 | 0a |
| AML 23 | t(8; 21) | dead | 28 | 0a |
| AML 77 | inv(16) | dead | 44 | 0a |
| AML 28 | normal karyotype | dead | 78 | 0a |
| AML 91 | normal karyotype | dead | 94 | 0a |
| AML 64 | normal karyotype | dead | 96 | 0a |
| AML 7 | normal karyotype | dead | 134 | 0a |
| AML 22 | normal karyotype | dead | 154 | 0a |
| AML 73 | inv(16) | dead | 177 | 0a |
| AML 11 | normal karyotype | dead | 204 | 0a |
| AML 40 | normal karyotype | dead | 215 | 0a |
| AML 111 | t(9; 11) | dead | 278 | 0a |
| AML 110 | normal karyotype | dead | 318 | 0a |
| AML 27 | normal karyotype | dead | 326 | 0a |
| AML 38 | t(8; 21) | dead | 334 | 0a |
| AML 88 | t(9; 11) | dead | 335 | 0a |
| AML 31 | +8sole | dead | 336 | 0a |
| AML 54 | other | dead | 346 | 0a |
| AML 36 | normal karyotype | dead | 374 | 0a |
| AML 37 | t(15; 17) | dead | 400 | 0a |
| AML 103 | inv(16) | dead | 429 | 0a |
| AML 15 | normal karyotype | dead | 483 | 0a |
| AML 74 | normal karyotype | dead | 511 | 0a |
| AML 85 | normal karyotype | dead | 1220 | 0a |
| AML 95 | t(15; 17) | alive | 365 | 0b |
| AML 99 | t(15; 17) | alive | 521 | 0b |
| AML 59 | other | alive | 724 | 0b |
| AML 83 | t(9; 11) | alive | 744 | 0b |
| AML 69 | t(9; 11) | alive | 748 | 0b |
| AML 2 | t(15; 17) | alive | 801 | 0b |
| AML 33 | t(15; 17) | alive | 836 | 0b |
| AML 68 | t(9; 11) | alive | 1053 | 0b |
| AML 86 | t(15; 17) | alive | 1212 | 0b |
| AML 101 | t(15; 17) | alive | 1352 | 0b |
| AML 119 | t(15; 17) | dead | 0 | 0b |
| AML 32 | +8sole | dead | 1 | 0b |
| AML 117 | t(15; 17) | dead | 1 | 0b |
| AML 104 | t(15; 17) | dead | 3 | 0b |
| AML 21 | t(9; 11) | dead | 21 | 0b |
| AML 106 | del(7q)/-7 | dead | 139 | 0b |
| AML 1 | complex karyotype | dead | 213 | 0b |
| AML 10 | normal karyotype | dead | 233 | 0b |
| AML 63 | del(7q)/-7 | dead | 281 | 0b |
| AML 60 | t(15; 17) | dead | 299 | 0b |
| AML 6 | del(7q)/-7 | dead | 336 | 0b |
| AML 29 | t(15; 17) | dead | 730 | 0b |

(a) The ES type was determined by using the gene list of 641 ES predictor genes selected at q ≦ 0.05 in the one-class SAM.

TABLE 6
Abbreviations

| Abbreviation | Full term |
|---|---|
| ES | embryonic stem |
| RNASEL | ribonuclease L (2′,5′-oligoisoadenylate |
| (HPC1) | synthetase-dependent)/hereditary prostate cancer 1 |
| ELAC2/HPC2 | elaC homolog 2 (E. coli)/hereditary prostate |
| | cancer 2 |
| GSTP1 | glutathione S-transferase pi |
| AMACR | alpha-methylacyl-CoA racemase |
| HPN | hepsin |
| PIM1 | pim-1 oncogene |
| EZH2 | enhancer of zeste homolog 2 |
| AZGP1 | alpha-2-glycoprotein 1, zinc |
| MUC1 | mucin 1, cell surface associated |
| SMD | Stanford Microarray Database |
| RNA | ribonuclear acid |
| DNA | dioxyribonuclear acid |
| cDNA | complementary dioxyribonuclear acid |
| SUID | Stanford Unique Identification Number |
| UID | unique Identification Number |
| R/G | red channel/green channel |
| GO | gene ontology |
| IMAGE | the Integrated Molecular Analysis of Genomes |
| | and their Expression |
| PSA | prostate specific antigen |
| RR | relative risk |
| SE | standard error |
| EBV | Epstein-Barr virus |
| ISH | in situ hybridization |
| AML | acute myeloid leukemia |
| H. pylori | Helicobacter pylori |
| SAM | significant analysis of microarrays |
| TF | transcriptional factor |
| t(15; 17) | translocation between chromosome 15 and |
| | chromosome 17 |
| del(7q) | deletion of the long arm of chromosome 7 |
| inv(16) | inversion of chromosome 16 |
| AML | acute myeloid leukemia. |
| NA | not available. |
| t(15; 17) | translocation between chromosome 15 and |
| | chromosome 17 |
| del(7q) | deletion of the long arm of chromosome 7 |
| inv(16) | inversion of chromosome 16 |
| F | female |
| M | male |

Note:
The gene symbols for all genes in this invention are given according to their standard symbol in the National Center for Biotechnology Information's gene database (http://www.ncbi.nlm.nih.gov/entrez/querv.fcgi?db=gene&cmd=search&term). For expressed sequence tag (EST) without gene symbol, the IMAGE clone ID or the UniGene cluster ID are given

1. A method of predicting the development of a cancer in a patient, comprising: (a) procuring a tumour tissue from the patient; (b) determining an expression pattern of a plurality of embryonic stem cell genes listed in Table 1; (c) comparing said expression pattern with a corresponding expression pattern of embryonic stem cell genes in tumour tissue of reference patients with known disease histories; (d) identifying the patient or patients with known disease histories whose expression pattern optimally matches the patient's expression pattern; (e) assigning, in a prospective manner, the disease history of said patient(s) to the patient in which the development of cancer shall be predicted.

2. The method of claim 1, wherein the determination of the expression pattern of said embryonic stem cell genes comprises that of a first group genes with high level of expression and that of a group of genes with a low level of expression, said first and second group of genes not comprising a third group of genes with intermediate levels of expression.

3. The method of claim 2, wherein the genes in at least one of the first group and the second group are consecutive in respect of their expression levels.

4. The method of claim 3, wherein the combined number of genes in the first and second groups is substantially smaller than the number of genes in the third group.

5. The method of claim 4, wherein said combined number is less than a fifth of the number of the genes in the third group.

6. The method of claim 5, wherein the combined number of genes in the first group and in the second group is from 500 to 750.

7. The method of claim 6, wherein the combined number of genes in the first and second group is from 600 to 680.

8. The method of claim 7, wherein the combined of genes in the first and second group is about 641.

9. The method of claim 2, wherein the genes of the first and second groups are identified by employing a q value of from 0.01 to 0.1 in a one class significant analysis of microarrays (SAM) on a centered embryonic stem cell gene dataset by which all genes are ranked according to their expression levels.

10. The method of claim 9, wherein the q value is from 0.025 to 0.075.

11. The method of claim 10, wherein the q value is about 0.05.

12. The method of claim 1, wherein the cancer is selected from the group consisting of prostate cancer, gastric cancer, lung cancer, leukemia, breast cancer, ovary cancer, brain tumor, soft tissue tumor, and kidney tumor.

13-19. (canceled)

20. A microarray comprising a fragment of embryonic stem cell gene DNA or RNA derived from a first group of embryonic stem cell genes with a high level of expression in a cancer tumor and of a second group of embryonic stem cell genes with a low level of expression in said cancer tumor but not comprising a fragment of embryonic stem cell gene DNA/RNA with an intermediate level of expression in said cancer tumor.

21. The microarray of claim 20, wherein the genes in at least one of the first group and the second group are consecutive in respect of their expression levels.

22. The microarray of claim 21, wherein the genes in the first and second groups are those ranked according to their expression levels by a one class significant analysis of microarrays (SAM) on a centered embryonic tumor stem cell gene dataset by employing a q value of from 0.01 to 0.1.

23. The microarray of claim 22, wherein the q value is from 0.025 to 0.075.

24. The microarray of claim 23, wherein the q value is about 0.05.

25. The microarray of claim 20, wherein the cancer is selected from the group consisting of prostate cancer, gastric cancer, lung cancer, leukemia, breast cancer, ovary cancer, brain tumor, soft tissue tumour, and kidney tumor.

26. (canceled)

27. A probe comprising a DNA, DNA fragment, DNA oligomer, DNA primer, RNA, RNA fragment, RNA oligomer of a first group of embryonic stem cell genes with high level of expression in a cancer tumor and of a second group of embryonic stem cell genes with a low level of expression in said cancer tumor but not comprising a DNA, DNA fragment, DNA oligomer, DNA primer, RNA, RNA fragment, RNA oligomer, respectively, of embryonic stem cell genes with an intermediate level of expression in said cancer tumor.

28. The probe of claim 27, wherein at least one of the genes in the first group and the second group are consecutive in respect of their expression levels.

29. The probe of claim 27, wherein the genes in the first and second groups are those ranked according to their expression levels by a one class significant analysis of microarrays (SAM) on a centered embryonic tumor stem cell gene dataset by employing a q value of from 00.1 to 0.1.

30. The probe of claim 29, wherein the q value is from 0.025 to 0.075.

31. The probe of claim 30, wherein the q value is about 0.05.

32. The probe of claim 27, wherein the cancer is selected from prostate cancer, gastric cancer, lung cancer, leukemia, breast cancer, ovary cancer, brain tumor, soft tissue tumor, and kidney cancer.

33-35. (canceled)

36. The method of claim 2, wherein the genes in the first and second groups constitute a fraction of the embryonic stem cell genes expressed in the tumor.

37. The method of claim 36, wherein said fraction is 20 per cent or less of the embryonic stem cell genes expressed in the tumor.

38-42. (canceled)
